(19) United States
(12) Patent Application Publication
(10) Pub. No.: US 2022/0354953 A1
(43) Pub. Date: Nov. 10, 2022

(54) PHOTODYNAMIC THERAPY COMPOSITIONS AND METHODS OF TREATMENT THEREIN

(71) Applicant: Pinnacle Biologics, Inc., Bannockburn, IL (US)

(72) Inventors: Glenn M. Kutschera, Bannockburn, IL (US); Thomas S. MANG, Bannockburn, IL (US)

(21) Appl. No.: 17/869,448

(22) Filed: Jul. 20, 2022

Related U.S. Application Data
(63) Continuation-in-part of application No. PCT/US2021/049928, filed on Jan. 10, 2021.
(60) Provisional application No. 63/223,835, filed on Jul. 20, 2021.
(60) Provisional application No. 63/223,839, filed on Jul. 20, 2021.

(51) Int. Cl. A61K 41/00; A61K 9/06; A61K 47/38; A61P 31/02

(52) U.S. Cl. CPC A61K 41/0071; A61K 9/06; A61K 47/38; A61P 31/02

(57) ABSTRACT

A composition and method directed to the treatment of sun exposed skin, wounds and microbial infections is provided.

### CROSS-REFERENCE TO RELATED APPLICATIONS

This application claims priority to PCT application No. PCT/US2021/049928, filed Sep. 10, 2021, and provisional application 63/223,835, filed Jul. 20, 2021, and provisional application 63/223,839, filed Jul. 20, 2021, the disclosures of which are herein incorporated by reference in their entirety for all purposes.

### BACKGROUND

Actinic keratoses (AKs) are common premalignant skin lesions that typically present as lesions on sun-exposed areas such as the face or forearms of affected persons. Actinic keratoses is also prevalent in the U.S. and worldwide. It has been estimated that 60% of predisposed persons aged >40 years have had at least 1 AK. AKs, if not treated, can develop into more serious diseases. For example, it has been estimated that AK develops into squamous cell carcinoma in 0.025% to 16% of cases. Accordingly, treatment of AKs when detected, is recommended.

Sun exposed skin can cause additional damaged skin, including pigment spots, liver spots, and general photo-damaged skin, which can also be treated. Treatments for AKs and sun exposed skin are available, including ablative procedures (surgery, laser ablation, curettage, cryotherapy) and Photodynamic therapy (PDT) using topical aminolaevulinic acid (5-ALA or mel-ALA). Photodynamic therapy (PDT) combines the use of non-toxic photosensitizers (PS) and low intensity visible non-thermal light that results in the production of cytotoxic species in the presence of oxygen. PDT has long been approved for use in the treatment of certain malignancies and other diseases in the United States (US) as well in many other countries. Early studies led to the development of porfimer sodium (Photofrin®, Concordia Laboratories Inc.), the first FDA approved PS for clinical use in the US. Since the development of this therapy, there have been numerous proposed and evaluated uses of PDT. While the combination of dyes and light activation has been applied to bacteria, viruses and fungi in vitro, there is currently no approved for use for treating bacteria, viruses and fungi in vivo, or other indications such as damaged skin caused by sun exposure, with this technology in the US, as such technology has been difficult to reproduce in vivo and in particular, for treatment of sun-exposed skin.

Accordingly, there remains a need to develop compositions and methods of use thereof for effective photodynamic treatment of damaged skin caused by sun exposure.

### SUMMARY OF THE INVENTION

In one aspect, the present disclosure provides pharmaceutical compositions comprising a photosensitizer and one or more gelling agents.

In some embodiments the present disclosure provides pharmaceutical topical formulations comprising porfimer sodium and one or more pharmaceutical acceptable excipients.

In some embodiments, the present disclosure provides methods for treating damaged skin, such as damaged skin caused by sun exposure in a subject, comprising administering to the subject in need thereof any pharmaceutical composition or formulation of as disclosed herein, wherein the composition is applied to the damaged or chronically sun-exposed skin; and light is applied to the damaged or chronically sun exposed skin. In a specific embodiment, the sun-exposed skin to be treated is selected from one or more of the group consisting of chronically sun exposed skin, wrinkling, pigment spots, liver spots, actinic keratosis, seborrheic keratosis photo-damaged skin, acne, warts, and psoriasis. In a specific embodiment, the sun-exposed skin to be treated is actinic keratosis.

In another embodiment, the subject does not suffer from pain when the light is applied to the sun exposed skin. In another embodiment, the subject does not suffer from pain per the visual analog scale of pain when the light is applied to the sun exposed skin.

In another embodiment, the subject does not develop edema or pruritis from treatment on the sun exposed skin. In another embodiment, the subject does not develop erythematous lesions from treatment on the sun exposed skin.

### BRIEF DESCRIPTION OF THE DRAWINGS

FIG. 1 shows porfimer sodium accumulation in epidermis for formulations Formulation 2, Formulation 3, Formulation 4, and Formulation 5 in the stability studies outlined in Example 3.

FIG. 2 shows the wavelength active range of porfimer sodium, PpIX and mTHPC.

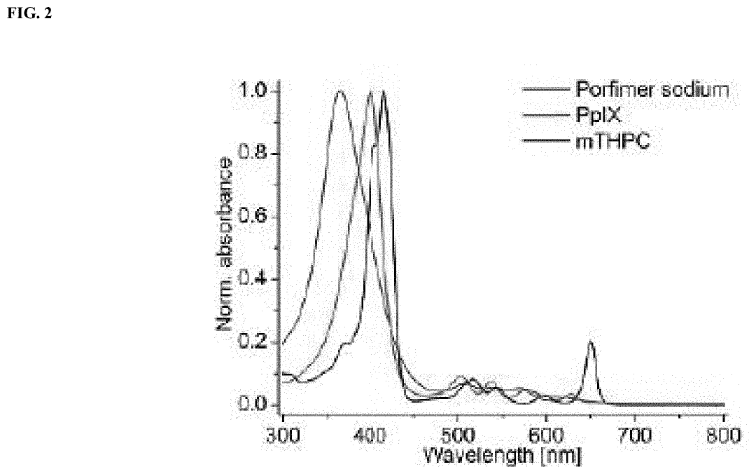

FIG. 3 shows the results of stability tests for formulations 7A, 7B, and 7C at 25° C./60% RH.

FIG. 4 shows the results of stability tests for formulations 7A, 7B, and 7C at 40° C./75% RH.

FIG. 5 shows the results of stability tests for formulations 7A, 7B, and 7C at 5° C.

FIG. 6 shows the results of stability tests for formulations 7D, 7E, and 7F at 25° C./60% RH.

FIG. 7 shows the results of stability tests for formulations 7D, 7E, and 7F at 40° C./75% RH.

FIG. 8 shows the results of stability tests for formulations 7D, 7E, and 7F at 5° C.

FIG. 9 depicts the design of the study described in Example 6.

FIG. 10 depicts the design of the study described in Example 7.

### DETAILED DESCRIPTION

In one aspect, the present disclosure provides compositions and methods for the photodynamic treatment of damaged skin caused by chronic sun exposure in a subject, including treating actinic keratosis, with a minimalization of adverse side effects, such as the minimization of pain, edema, pruritis and/or erythema.

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988), The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the verb “comprise” as is used in this description and in the claims and its conjugations are used in its non-limiting sense to mean that items following the word are included, but items not specifically mentioned are not excluded. The present disclosure may suitably “comprise”, “consist of”, or “consist essentially of”, the steps, elements, and/or reagents described in the claims.

Unless specifically stated or obvious from context, as used herein, the term “or” is understood to be inclusive. Unless specifically stated or obvious from context, as used herein, the terms “a”, “an”, and “the” are understood to be singular or plural.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

As disclosed herein, “photodynamic therapy” or “PDT” involves the use of a chemical photosensitizer or a nontoxic photoactivatable dye, visible non-thermal light, and reactive oxygen. The therapy is based on the energy (absorbed as light via the photosensitizer) transferred to the oxygen molecules producing extremely reactive mediation, such as singlet oxygen and superoxide, that are noxious to the cells. Photodynamic therapy requires a light source for triggering the photosensitizer with a low power visible light at a particular wavelength. Most of the optical photosensitizers are actuated by light of 380 nm to 850 nm wavelengths corresponding to a light penetration depth of 0.2 cm to 2.5 cm.

As disclosed herein, PDT uses “photosensitizers” or “PS” that are activated by absorption of visible light or near infrared light to initially form the excited singlet state, followed by transition to the long-lived excited triplet state. This triplet state can undergo photochemical reactions in the presence of oxygen to form reactive oxygen species (including singlet oxygen) that can destroy pathogenic microbes and/or treat damaged skin. The dual-specificity of PDT relies on accumulation of the PS in infected tissue and also on localized light delivery. As disclosed herein, PSs can include but are not limited to porphyrin based photosensitizers and tetrapyrrole structures such as porphyrins, chlorins (HPPH; NPe6; Temoporfrin (Foscan), mTHPC)), and porphysomes as in: pyropheophorbide nanovesicles including, bacteriochlorophyll porphysomes, zinc pyropheophorbide porphysomes and pyropheophyorbide porphysomes, and chlorin-like compounds (benzoporphyrin; Verteporfin, bacteriochlorins and phthalocyanines, purpurins (tin ethyl etiopurpurin); Metalloporphyrins (Texaphyrins); Pheophorbides (TOOKAD); Protoporphyrins (Levulan, Metvix, 5-ALA (PpIX)) and nonporphyrin based photosensitizers including phenothiazinium salts such as Methylene Blue, Toluidine Blue, Nile Blue, Cyanines, hypericin and Chalcogenpyrilium dyes; PPA904; benzophenothiazinium dye EtNBS; PS can also include the xanthene class of fluorescent dyes that includes fluorescein and Rose Bengal; Fullerenes (C60 fullerene coupled to polar diserinol groups or quartemary pyrrolidinium groups) Also Squaraogenines, BODIPY (boron-dipyrromethene) dye, Phenalenones; Hypericin, Hypocrellin, Riboflavin, Curcumin, Titanium dioxide. As used herein, the preferred PS is porfimer sodium (Photofrin®).

As disclosed herein, “potentiator of PDT” refers to for example, potassium iodide (KI) which has a potentiating effect on PDT. For example, addition of KI to a mixture of microbial cells and a PS that is subsequently excited by light can give many log units of additional killing of microbes. Addition of KI to porfimer sodium allows broad-spectrum PDT. As disclosed herein, a potentiator of PDT can include inorganic salts, such as sodium azide, sodium thiocyanate, sodium bromide and potassium iodide, sodium and iodide, and potassium selenocyanate (KSeCN). In some embodiments, the potentiator of PDT is potassium iodide (KI).

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “managing” includes therapeutic treatments as defined above. Managing includes achieving a steady state level of infection as determined by known methods in the art. The steady state can include evaluation of one or more of the severity of the infection(s), the size and location of the infection(s), the number of different microbial pathogens present in the infection(s), the level of antibiotic tolerant or resistant microbial pathogens, the degree of response to treatment, such as with a PS composition disclosed herein, the degree of biofilm formation and reduction, and the side effects experienced by the subject. During management of an infection, the infection may fluctuate from increasing to lessening in severity, in the amount or extent of infection, amount of side effects experienced by the subject, or other subject outcome indicia. Over a period of time, such as days, months, or years, the degree of management of the infection can be determined by evaluation of the above factors to assess whether the clinical course of infection has improved, is bacteriostatic, or has worsened. In some embodiments, managing an infection include successful treatment of microbial pathogen(s) that are otherwise drug tolerant or drug resistant.

As disclosed herein, “sun-exposed skin” means any skin or body area on a subject that has noticeable chronic sun exposure, sun-damaged skin such as wrinkling, pigment spots, sun-induced moles, seborrheic keratoses, and/or actinic keratoses.

The term “lessen the severity” of damage refers to an improvement in the clinical course of damaged skin or chronically sun-exposed skin on any measurable basis. Such basis can include measurable indices such as reducing the extent of damaged skin, such as actinic keratosis, or photo-damaged skin. In some embodiments, lessening the severity of damaged skin such as actinic keratosis, or photo-damaged or sun-exposed skin is determined by measuring an improvement in clinical signs and symptoms. For example, lessening the severity of actinic keratosis can be for example, lessening rough and/or scaly skin patches. In some embodiments, for example, lessening the severity involves halting a steady decline in symptoms, resulting in the subject entering successful management of the symptoms. In other embodiments, lessening the severity can result in substantial to complete treatment of the sun-exposed skin.

An “effective amount”, as used herein, refers to an amount that is sufficient to achieve a desired biological effect. A “therapeutically effective amount”, as used herein refers to an amount that is sufficient to achieve a desired therapeutic effect. For example, a therapeutically effective amount can refer to an amount that is sufficient to improve at least one sign or symptom of damaged or sun-exposed skin.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

A “response” to a method of treatment can include, among other things, a decrease in or amelioration of negative signs and symptoms, a decrease in the progression of an infection or symptoms thereof, an increase in beneficial symptoms or clinical outcomes, a lessening of side effects, stabilization of the disease or indication, and partial or complete remedy of disease or indication.

“Antibiotic susceptibility or sensitivity” refers to whether a bacteria will be successfully treated by a given antibiotic. Similarly, “Antifungal susceptibility or sensitivity” refers to whether a fungi will be successfully treated by a given antibiotic. Testing for susceptibility can be performed by methods known in the art such as the Kirby-Bauer method, the Stokes method and Agar Broth dilution methods. The effectiveness of an antibiotic in killing the bacteria or preventing bacteria from multiplying can be observed as areas of reduced or stable amount, respectively, of bacterial growth on a medium such as a wafer, agar, or broth culture.

“Antimicrobial resistance” refers to the ability of a microbe to resist the effects of medication that once could successfully treat the microbe. Microbes resistant to multiple antimicrobials are called multidrug resistant (MDR). Resistance arises through one of three mechanisms: natural resistance in certain types of bacteria, genetic mutation, or by one species acquiring resistance from another. Mutations can lead to drug inactivation, alteration of the drug's binding site, alteration of metabolic pathways and decreasing drug permeability.

As used herein, the terms “antibacterial activity”, “antifungal activity” and “antimicrobial activity”, with reference to a PS composition of the present disclosure, refers to the ability to kill and/or inhibit the growth or reproduction of a particular microorganism. In certain embodiments, antibacterial or antimicrobial activity is assessed by culturing bacteria, e.g., Gram-positive bacteria (e.g., S. aureus). Gram-negative bacteria (e.g., Moraxella catarrhalis) or atypical bacteria not classified as either Gram-positive or Gram-negative, or fungi (e.g., C. albicans) according to standard techniques (e.g., in liquid culture or on agar plates), contacting the culture with a PS composition of the present disclosure and monitoring cell growth after said contacting. For example, in a liquid culture, bacteria may be grown to an optical density (“OD”) representative of a mid-point in exponential growth of the culture, the culture is exposed to one or more concentrations of one or more PS compounds of the present disclosure, or variants thereof, and the OD is monitored relative to a control culture. Decreased OD relative to a control culture is representative of antibacterial activity (e.g., exhibits lytic killing activity). Similarly, bacterial colonies can be allowed to form on an agar plate, the plate exposed to a PS composition of the present disclosure, or variants thereof, and subsequent growth of the colonies evaluated related to control plates. Decreased size of colonies, or decreased total numbers of colonies, indicate antibacterial activity.

“Biofilm” refers any syntrophic consortium of microorganisms in which cells stick to each other and often also to a surface. These adherent cells become embedded within a slimy extracellular matrix that is composed of extracellular polymeric substances (EPS). Upon formation of biofilms, microbial resistance to antibiotics is up to 1000 times greater compared to that of planktonic bacteria. Bacterial aggregates are clusters of laterally aligned cells can initiate biofilm development, which has a more complex and denser 3-D structure. In some embodiments, the biofilm may comprise one or more species of bacteria (e.g., Moraxella catarrhalis and Staphylococcus aureus) and/or one or more different phyla (e.g., bacteria and fungi).

The term “infection” is used herein in its broadest sense and refers to any infection, such as caused by a microorganism bacterial infection or fungal infection. Examples of such infections can be found in a number of well-known texts such as “Medical Microbiology” (Greenwood, D., Slack, R., Peutherer, J., Churchill Livingstone Press, 2002); “Mims' Pathogenesis of Infectious Disease” (Minis, C., Nash, A., Stephen, J., Academic Press, 2000); “Fields” Virology. (Fields, B N, Knipe D M, Howley, P M, Lippincott Williams and Wilkins, 2001); and “The Sanford Guide To Antimicrobial Therapy,” 26th Edition, JP Sanford et al. (Antimicrobial Therapy, Inc. 1996), which is incorporated by reference herein.

As used herein, the terms “prophylactic agent” and “prophylactic agents” refer to an agent, such as a PS composition of the present disclosure, which can be used in the prevention, management, or control of one or more signs and symptoms of the damaged area, including a disease or disorder, in particular, disease or disorder associated with sun-exposed skin such as actinic keratosis.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of other active agents would either completely lack other active agents, or so nearly completely lack other active agents that the effect would be the same as if it completely lacked other active agents. In other words, a composition that is “substantially free of an ingredient or element or another active agent may still contain such an item as long as there is no measurable effect thereof.

As used herein, “weight for weight” or “weight by weight” or “w/w”, refers to the proportion of a particular substance within a mixture, as measured by weight or mass. “% wt” as used herein refers to the percent of the total weight of the composition.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The present invention relates to compositions, including pharmaceutical compositions, comprising a photosensitizer and one or more pharmaceutically acceptable excipients, and methods related to administering said compositions to a subject for treating a damaged skin caused by sun exposure in a subject.

### Pharmaceutical Compositions

In some embodiments, the present disclosure provides a pharmaceutical composition comprising a photosensitizer, one or more gelling agents, and one or more pharmaceutically acceptable excipients or carriers. In some embodiments of the pharmaceutical compositions disclosed herein, the pharmaceutical composition comprises a photosensitizer, a gelling agent, one or more permeation enhancers, a humectant, a stabilizer, a solubilizer, and/or a preservative. In some embodiments, the pharmaceutical composition may also include one or more solvents, buffers, bodifying agents, antioxidants, stabilizers and surfactants. In another embodiments, the pharmaceutical composition comprises one or more photosensitizer, a gelling agent, one or more permeation enhancers, a humectant, a solubilizer, a preservative and/or a potentiator.

In another embodiment, the present disclosure provides a pharmaceutical composition comprising a photosensitizer one or more gelling agents, and one or more pharmaceutically acceptable excipients or carriers. In some embodiments of the pharmaceutical compositions disclosed herein, the pharmaceutical composition comprises a photosensitizer, a gelling agent, one or more permeation enhancers, a humectant, a stabilizer, a solubilizer, and/or a preservative. In some embodiments, the pharmaceutical composition may also include one or more solvents, buffers, bodifying agents, antioxidants, and surfactants.

In some embodiments of the pharmaceutical compositions disclosed herein, the photosensitizer is selected from one or more of the group consisting of porphyrin based photosensitizers and tetrapyrrole structures such as porphyrins, chlorins (HPPH; NPe6; Temoporfrin (Foscan), mTHPC)), and porphysomes as in: pyropheophorbide nanovesicles including, bacteriochlorophyll porphysomes, zinc pyropheophorbide porphysomes and pyropheophyorbide porphysomes, and chlorin-like compounds (benzoporphyrin; Verteporfin, bacteriochlorins and phthalocyanines, purpurins (tin ethyl etiopurpurin); Metalloporphyrins (Texaphyrins); Pheophorbides (TOOKAD); Protoporphyrins (Levulan, Metvix, 5-ALA (PpIX)) and nonporphyrin based photosensitizers including phenothiazinium salts such as Methylene Blue, Toluidine Blue, Nile Blue, Cyanines, hypericin and Chalcogenpyrilium dyes; PPA904; benzophenothiazinium dye EtNBS; PS can also include the xanthene class of fluorescent dyes that includes fluorescein and Rose Bengal; Fullerenes (C60 fullerene coupled to polar diserinol groups or quartemary pyrrolidinium groups) Also Squaraogenines, BODIPY (boron-dipyrromethene) dye, Phenalenones; Hypericin, Hypocrellin, Riboflavin, Curcumin, Titanium dioxide. As used herein, the preferred PS is porfimer sodium (Photofrin®).

In some embodiments of the pharmaceutical compositions disclosed herein, the porfimer sodium is in an amount ranging from about 0.01% wt to about 1.0% wt; or about 0.05% wt to about 0.7% wt; or about 0.1% wt to about 0.5% wt; or about 0.15% wt to about 0.3% wt. In some embodiments, the porfimer sodium is an amount ranging from 0.01% wt to 1.0% wt; or 0.05% wt to 0.7% wt; or 0.1% wt to 0.5% wt; or 0.15% wt to 0.3% wt. In some embodiments of the pharmaceutical compositions disclosed herein, the porfimer sodium is present in an amount ranging of from about 0.01% wt, about 0.02% wt, 0.03% wt, 0.04% wt, about 0.05% wt, about 0.06% wt, about 0.07% wt, about 0.08% wt, about 0.09% wt, about 0.1% wt, about 0.2% wt, about 0.3% wt, about 0.4% wt, about 0.5% wt, about 0.6% wt, about 0.7% wt, about 0.8% wt, about 0.9% wt to about 1.0% wt, including all values and subranges therebetween. In some embodiments, the porfimer sodium is in an amount ranging from about 0.01% wt to about 1.0% wt; or about 0.05% wt to about 0.7% wt; or about 0.1% wt to about 0.5% wt. In some embodiments, the porfimer sodium is an amount ranging from 0.01% wt to 1.0% wt; or 0.05% wt to 0.7% wt; or 0.1% wt to 0.5% wt. In some embodiments, the porfimer sodium is an amount ranging from about 0.05% wt to about 0.15% wt; or about 0.15% wt to 0.25% wt; or 0.4% wt to 0.6% wt.

Gelling agents may be added to the pharmaceutical compositions of the present invention. Gelling agents are any suitable substance that is used to modify the viscosity of the composition. For example, gelling agents may be highly crosslinked or otherwise possess strong intermolecular interactions to increase the cohesion of the composition. Various gelling agents can be employed including, for example and without limitation, sugars or sugar derived alcohols, such as mannitol, sorbitol, and the like, starch and starch derivatives, cellulose derivatives, such as microcrystalline cellulose, sodium cahoxymethyl cellulose, methylcellulose, ethyl cellulose, hydroxyethyl cellulose, hydroxypropyl cellulose, and hydroxypropyl methylcellulose, attapulgites, bentonites, dextrins, alginates, carrageenan, gum tragacanth, gum acacia, guar gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof, etc. Examples of suitable gelling agents include hydroxy propyl cellulose, acrylic acids such as Carbopol 980, and lecithins such as Lecithin-PLO.

For topical application to the skin, the pharmaceutical compositions of the present disclosure may be combined with one or a combination of gelling agents for topical formulations, which can include, but are not limited to, an aqueous liquid, an alcohol base liquid, a water soluble gel, a lotion, an ointment, a nonaqueous liquid base, a mineral oil base, a blend of mineral oil and petrolatum, lanolin, liposomes, proteins carriers such as serum albumin or gelatin, powdered cellulose carmel, carbomer polymers such as carbomer homopolymers, carbomer copolymers, including Carbopol® polymers, and combinations thereof. Carbopol® polymers are polymers of acrylic acid cross-linked e.g., with polyalkenyl ethers or divinyl glycol. In some embodiments, the Carbopol® polymer is Carbopol® 71G, Carbopol® 971P, Carbopol® 974P, Carbopol® 980, Carbopol® 981, Carbopol® 5984, Carbopol® 934, Carbopol® 934P, Carbopol® 940, Carbopol® 941, and Carbopol® 1342. The compositions of the present invention comprise semi-solid and gel-like vehicles that include a polymer thickener, water, preservatives, active surfactants or emulsifiers, antioxidants, and a solvent or mixed solvent system. The solvent or mixed solvent system is important to the formation of the microparticulate to dissolved pharmaceutical ratio. The formation of the microparticulate, however, should not interfere with the ability of the polymer thickener or preservative systems to perform their functions.

Polymer thickeners that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL®” (B. F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.).

In some embodiments of the pharmaceutical compositions disclosed herein, the gelling agent is selected from one or more of the group consisting of a carbomer, crosslinked, polyacrylic acid, lecithin, hydroxyethyl cellulose, hydroxypropyl cellulose, and hydroxypropyl methylcellulose. In some embodiments, the gelling agent is a polymer of acrylic acid cross-linked with polyalkenyl ethers or divinyl glycol. In some embodiments, the gelling agent is a carbomer. In some embodiments, the carbomer is Carbopol® 71G, Carbopol® 971P, Carbopol® 974P, Carbopol® 980, Carbopol® 981, Carbopol® 5984, Carbopol® 934, Carbopol® 934P, Carbopol® 940, Carbopol® 941, and/or Carbopol® 1342.

In some embodiments of the pharmaceutical compositions disclosed herein, the gelling agent is in an amount ranging from about 0.5% wt to about 3.0% wt, or from about 0.7% wt to about 2.0% wt, or from about 1.0% wt to about 1.75% wt. In some embodiments, the gelling agent is in an amount ranging from 0.5% wt to 3.0% wt, or from 0.7% wt to 2.0% wt, or from 1.0% wt to 1.75% wt, or from 1.1% wt to 1.6% wt, or from 1.2% wt to 1.5% wt including all values and subranges therebetween. In some embodiments, the gelling agent is in an amount ranging from about 0.5% wt to about 3.0% wt, or from about 0.7% wt to about 2.0% wt, or from about 0.5% wt to about 1.5% wt. In some embodiments, the gelling agent is in an amount ranging from 0.5% wt to 3.0% wt, or from 0.7% wt to 2.0% wt, or from 0.5% wt to 1.5% wt.

In some embodiments, the pharmaceutical composition or pharmaceutical topical formulation further comprises one or more permeation enhancers.

As defined herein, a “permeation enhancer” is taken to mean any substance which acts as a skin penetrant and enhances the ability for the active agent to pass through the epidermal tissue into dermal tissue, or through dermal tissue. A permeation enhancer may thus be for example a penetration enhancer. In some embodiments, the permeation enhancer may be one or more of an alcohol, amide, fatty acid, ester, ether alcohol, surfactant, phospholipid, pyrrolidone or a terpene. In a specific embodiment, the permeation enhancer may be one or more of ethanol, isopropyl alcohol, decanol, octanol, propylene glycol, polyethylene glycol, Azone® (1-dodecylazacycloheptan-2-one or laurocapram, lauric acid, oleic acid, linoleic acid, ethyl acetate, butyl acetate, methyl acetate, isopropyl myristate, isopropyl palmitate, transcutol such as diethylene glycol monoethyl ether (DEGEE), sodium lauryl sulphate, benzalkonium chloride, cetylpyridinium chloride, cetyltrimethylammonium bromide; polysorbates (Tween® 20, Tween® 80, etc.), dodecyl betaine, dimethyl sulfoxide (DMSO), decylmethyl sulphoxide (DCMS), D-Limonene, L-menthol, 1,8-Cineole, N-methyl-1-2-pyrrolidone (NMP), 2-pyrrolidone (2P), N-methyl-1-2-pyrrolidone (NMP), 4-decyloxazolidin-2-one, phosphatidylcholine, acid phosphatase, papain, and phospholipase C.

Permeation enhancer may have also act as solvents, or have solvating effects, may act as a surface surfactant enhancer, and/or act as an emulsifier. Permeation enhancers that may be used include those known to one skilled in the art, polyols and esters including, glycol esters (e.g., polyethylene glycol, polyethylene glycol monolaurate) and butanediol; sulfoxides, including dimethyl sulfoxide and decylmethyl sulfoxide; ethers, including diethylene glycol monoethyl ether (e.g., Transcutol® P); fatty acids, including lauric acid, oleic acid, and valeric acid; fatty acid esters, including isopropyl myristate, isopropyl palmitate, methyl propionate, and ethyl oleate; nitrogenous compounds including urea, dimethyl acetamide, dimethylformamide 2-pyrrolidone, ethanolamine, methyl-2-pyrrolidone, diethanolamine, and triethanolamine; terpenes; alkanones; organic acids, including salicylic acid, citric acid, and succinic acid; azones, polysorbates, alcohols, and any mixtures thereof. Further, one or more surfactants can also be used as a permeation or permeation enhancer. In some embodiments, the permeation enhancer is selected from one or more of the group consisting of propylene glycol, polyethylene glycol of average molecular weight from 200 to 4000, diethylene glycol monoethyl ether, Transcutol P, Polysorbate 80, polyoxylglycerides, Labrasol®, diethyl sebacate, diisopropyl adipate, dimethyl isosorbide, dimethyl sulfoxide, ethanol, Tween 80, Laureth-4, butanediol, polyethylene glycol monolaurate, diethylene glycol monoethyl ether, dimethyl sulfoxide, decylmethyl sulfoxide, lauric acid, oleic acid, valeric acid, isopropyl myristate, isopropyl palmitate, methyl propionate, ethyl oleate, and oleic acid. Suitable examples of permeation enhancers include hexylene glycol, propylene glycol SR, polyethylene glycol 400 SR, polyethylene glycol 300 LA, diethylene glycol monoethyl ether, and Polysorbate 80 SR.

In some embodiments, the permeation enhancer is selected from one or more of the group consisting of diethylene glycol monoethyl ether, dimethyl sulfoxide, propylene glycol, polyethylene glycol, and the various forms, molecular weights and grades therein. In a specific embodiment, the permeation enhancer is selected from one or more of the group consisting of propylene glycol SR, polyethylene glycol 400 SR, polyethylene glycol 300 LA, diethylene glycol monoethyl ether, dimethyl sulfoxide, and Polysorbate 80 SR. In some embodiments, the permeation enhancer is selected from one propylene glycol SR, dimethyl sulfoxide, and diethylene glycol monoethyl ether.

In some embodiments of the pharmaceutical compositions disclosed herein, the one or more permeation enhancers is in an amount ranging from about 0.5% wt to about 90% wt, about 0.5% wt to about 80% wt, about 0.5% wt to about 70% wt, or from about 1% wt to about 60% wt, or from about 10% wt to about 60% wt, or from about 5% wt to about 30% wt, or from about 15% wt to about 30% wt. In some embodiments, the permeation enhancer is in an amount ranging from 0.5% wt to 50% wt, or from 10% wt to 40% wt, or from 15% wt to 30% wt.

In embodiment, more than one permeation enhancer is provided in a single composition with the ranges as disclosed directly above. In a specific embodiment, four permeations are provided in a single composition. In another embodiment, three permeation enhancers are provided in a single composition. In another embodiment, two permeation enhancers are provided in a single composition. In a specific embodiment, the more than one permeation enhancers may range from about 55% wt to about 85% wt, from about 65% wt to about 80% wt, or from about 70% wt to about 80% wt. In another embodiment, the single composition may comprise a single permeation enhancer. In one embodiment, the single permeation enhance may range from about 0.5% wt to about 60% wt, about 10% wt to about 50% wt, about 15% wt to about 45% wt, or from about 20% wt to about 40% wt, or from about 25 wt to about 40% wt. In one embodiment, at least one of the permeation enhancers is DMSO.

In some embodiments, the compositions disclosed herein comprise a solubilizer. In some embodiments of the pharmaceutical compositions disclosed herein, the solubilizer is in an amount ranging from 1.0% wt to about 5% wt, or about 1% wt to about 3% wt, or about 2% wt. In some embodiments, the solubilizer is in an amount of about 1.0% wt, or about 2% wt, or about 3% wt, or about 4% wt, or about 5% wt.

In some embodiments of the compositions disclosed herein, the permeation enhancer is in an amount ranging from about 0.5% wt, about 1% wt, about 5% wt, about 10% wt, about 11% wt, about 12% wt, about 13% wt, about 14% wt, about 15% wt, about 16% wt, about 17% wt, about 18% wt, about 19% wt, about 20% wt, about 21% wt, about 22% wt, about 23% wt, about 24% wt, about 25% wt, about 26% wt, about 27% wt, about 28% wt, about 29% wt, about 30% wt, about 31% wt, about 32% wt, about 33% wt, about 34% wt, about 35% wt, about 36% wt, about 37% wt, about 38% wt, about 39% wt, about 40% wt, about 41% wt, about 42% wt, about 43% wt, about 44% wt, about 45% wt, about 46% wt, about 47% wt, about 48% wt, about 49% wt, about 50% wt, about 51% wt, about 52% wt, about 53% wt, about 54% wt, about 55% wt, about 56% wt, about 57% wt, about 58% wt, about 59% wt, about 60% wt, about 61% wt, about 62% wt, about 63% wt, about 64% wt, about 65% wt, about 66% wt, about 67% wt, about 68% wt, about 69% wt, about 70% wt, including all subranges therebetween. In some embodiments, the permeation enhancer is in an amount ranging from about 1.0% wt to about 80% wt, or about 15% wt to about 75% wt. In some embodiments, the pharmaceutical compositions or formulations disclosed herein comprise dimethyl sulfoxide in an amount ranging from 1.0% wt to about 60% wt, including about 1.0% wt, about 5.0% wt, about 10% wt, about 15% wt, about 20% wt, about 25% wt, about 30% wt, about 35% wt, about 40% wt, about 45% wt, about 50% wt, about 55% wt, about 60% wt, or any sub-ranges and values therebetween. In some embodiments, the pharmaceutical compositions or formulations disclosed herein comprise diethylene glycol monoethyl ether in an amount ranging from 1.0% wt to about 60% wt, including about 1.0% wt, about 5.0% wt, about 10% wt, about 15% wt, about 20% wt, about 25% wt, about 30% wt, about 35% wt, about 40% wt, about 45% wt, about 50% wt, about 55% wt, about 60% wt, or any sub-ranges and values therebetween. In some embodiments, the pharmaceutical compositions or formulations disclosed herein comprise diethylene glycol monoethyl ether in an amount ranging from 1.0% wt to about 60% wt and dimethyl sulfoxide in an amount ranging from 1.0% wt to about 60% wt. In some embodiments, the pharmaceutical compositions or formulations disclosed herein comprise diethylene glycol monoethyl ether in an amount of about 20% wt to 40% wt and dimethyl sulfoxide in an amount of about 10% wt to about 50% wt. In some embodiments, the pharmaceutical compositions or formulations disclosed herein comprise about 30% wt of diethylene glycol monoethyl ether and about 20% wt or about 40% wt of dimethyl sulfoxide.

In some embodiments, the compositions disclosed herein comprise a potentiator of PDT. In a specific embodiment, the potentiator of PDT may be one or more inorganic salts, such as sodium azide, sodium thiocyanate, sodium bromide, potassium iodide (KI), sodium iodide, and potassium selenocyanate (KSeCN). In a specific embodiment, the potentiator of PDT is potassium iodide (KI). In some embodiments of the pharmaceutical compositions disclosed herein, the concentration of the potentiator of PDT (e.g., KI) is in an amount ranging from about 0.1 mM to about 1 M. In some embodiments, the potentiator of PDT the potentiator of PDT (e.g., KI) is in an amount ranging from about 10 mM to about 100 mM. For example, in some embodiments, the potentiator of PDT (e.g., KI) is in an amount ranging from about 10 mM, or about 20 mM, or about 30 mM, or about 40 mM, or about 50 mM, or about 60 mM, or about 70 mM, or about 80 mM, or about 90 mM, or about 100 mM or about 110 mM, or about 120 mM, or about 130 mM, or about 140 mM, or about 150 mM, or about 160 mM, or about 170 mM, or about 180 mM, or about 190 mM, or about 200 mM, or about 210 mM, or about 220 mM, or about 230 mM, or about 240 mM, or about 250 mM, or about 260 mM, or about 270 mM, or about 280 mM, or about 290 mM, or about 300 mM, or about 310 mM, or about 320 mM, or about 330 mM, or about 340 mM, or about 350 mM, or about 360 mM, or about 370 mM, or about 380 mM, or about 390 mM, or about 400 mM, or about 410 mM, or about 420 mM, or about 430 mM, or about 440 mM, or about 450 mM, or about 460 mM, or about 470 mM, or about 480 mM, or about 490 mM, to about 500 mM, including all sub-ranges and values therebetween. In some embodiments, the potentiator of PDT the potentiator of PDT (e.g., KI) is in an amount of about 100 mM. In a specific embodiment, the potentiator of PDT in the amounts or ranges herein is KI.

In some embodiments, the potentiator of PDT the potentiator of PDT (e.g., KI) is in an amount ranging from about 0.01% wt to about 5% wt. For example, in some embodiments, the potentiator of PDT (e.g., KI) is in an amount ranging from about 0.1% wt, about 0.2% wt, about 0.3% wt, about 0.4% wt, about 0.5% wt, about 0.6% wt, about 0.7% wt, about 0.8% wt, about 0.9% wt, about 1% wt, about 1.1% wt, about 1.2% wt, about 1.3% wt, about 1.4% wt, about 1.5% wt, about 1.6% wt, about 1.7% wt, about 1.8% wt, about 1.9% wt, about 2.0% wt, about 2.1% wt, about 2.2% wt, about 2.3% wt, about 2.4% wt, about 2.5 wt, about 2.6% wt, about 2.7% wt, about 2.8% wt, about 2.9% wt, about 3.0% wt, about 3.1% wt, about 3.2% wt, about 3.3% wt, about 3.4% wt, about 3.5% wt, about 3.6% wt, about 3.7% wt, about 3.8% wt, about 3.9% wt, about 4.0% wt, about 4.2% wt, about 4.4% wt, about 4.6% wt, about 4.8% wt, about 5.0% wt, about 5.5% wt, about 6.0% wt, about 6.5% wt, about 7.0% wt, about 7.5% wt, about 8.0% wt, about 8.5% wt, about 9.0% wt, about 9.5% wt, about 10.0% wt, about 10.5% wt, about 11.0% wt, about 11.5% wt, about 12.0% wt, about 12.5% wt, about 13.0% wt, about 13.5% wt, about 14.0% wt, about 14.5% wt, to about 15.0% wt, including all sub-ranges and values therebetween. In some embodiments, the potentiator of PDT (e.g., KI) is in an amount ranging from about 0.5% wt to about 5% wt, or from about 1% wt to about 2% wt. In a specific embodiment, the potentiator of PDT in the amounts or ranges herein is KI.

In some embodiments of the pharmaceutical compositions disclosed herein, the potentiator of PDT is selected from the group consisting of sodium azide, sodium thiocyanate, sodium bromide, potassium iodide, and sodium iodide. In some embodiments, the potentiator of PDT is potassium iodide (KI).

In some embodiments, the pharmaceutical composition or pharmaceutical topical formulation further comprises one or more surfactants. In some embodiments, the surfactant is a polysorbate, such as Polysorbate 80. Furthermore, other surfactants may be used instead of Polysorbate 80; for example, sulfates such as sodium lauryl sulfate, and the related alkyl-ether sulfates sodium lauryl ether sulfate, and sodium myreth sulfate; polyethylene glycol, the fraction size of polyethylene glycol may be varied. Other suitable surfactants include alkyl-aryl ether phosphates, alkyl ether phosphates, and carboxylates, such as sodium stearate, or non-ionic surfactants such as ethoxylates, fatty acid esters of polyhydroxy compounds, fatty acid esters of sorbitol, fatty acid esters of sucrose, and alkyl polyglucosides.

In some embodiments of the pharmaceutical compositions disclosed herein, the surfactant is in an amount ranging from 0.01% wt to about 5% wt, including about 0.01%, about 0.05%, about 0.1% wt, about 0.2% wt, about 0.3% wt, about 0.4% wt, about 0.5% wt, about 0.6% wt, about 0.7% wt, about 0.8% wt, about 0.9% wt, about 1% wt, about 1.1% wt, about 1.2% wt, about 1.3% wt, about 1.4% wt, about 1.5% wt, about 1.6% wt, about 1.7% wt, about 1.8% wt, about 1.9% wt, about 2.0% wt, about 2.1% wt, about 2.2% wt, about 2.3% wt, about 2.4% wt, about 2.5% wt, about 2.6% wt, about 2.7% wt, about 2.8% wt, about 2.9% wt, about 3.0% wt, about 3.1% wt, about 3.2% wt, about 3.3% wt, about 3.4% wt, about 3.5% wt, about 3.6% wt, about 3.7% wt, about 3.8% wt, about 3.9% wt, about 4.0% wt, about 4.2% wt, about 4.4% wt, about 4.6% wt, about 4.8% wt, to about 5.0% wt, including all sub-ranges and values therebetween. In some embodiments, the solvent is in an amount ranging from about 0.5% wt to about 1.5% wt, or about 1% wt.

In some embodiments, the pharmaceutical composition or pharmaceutical topical formulation further comprises one or more solvents. Among the acceptable solvents that may be employed are water, Ringer's solution and isotonic sodium chloride solution. In addition, non-aqueous solvents may be employed. Examples of non-aqueous solvents suitable for use in the present application include, but are not limited to, propylene glycol, hexylene glycol, polyethylene glycol, vegetable oils such as olive oil, and injectable organic esters such as ethyl oleate. In some embodiments, the solvent is hexylene glycol.

In some embodiments of the pharmaceutical compositions disclosed herein, the solvent is in an amount ranging from 0.01% wt to about 5% wt, including about 0.01%, about 0.05%, about 0.1% wt, about 0.2% wt, about 0.3% wt, about 0.4% wt, about 0.5% wt, about 0.6% wt, about 0.7% wt, about 0.8% wt, about 0.9% wt, about 1% wt, about 1.1% wt, about 1.2% wt, about 1.3% wt, about 1.4% wt, about 1.5% wt, about 1.6% wt, about 1.7% wt, about 1.8% wt, about 1.9% wt, about 2.0% wt, about 2.1% wt, about 2.2% wt, about 2.3% wt, about 2.4% wt, about 2.5% wt, about 2.6% wt, about 2.7% wt, about 2.8% wt, about 2.9% wt, about 3.0% wt, about 3.1% wt, about 3.2% wt, about 3.3% wt, about 3.4% wt, about 3.5% wt, about 3.6% wt, about 3.7% wt, about 3.8% wt, about 3.9% wt, about 4.0% wt, about 4.2% wt, about 4.4% wt, about 4.6% wt, about 4.8% wt, to about 5.0% wt, including all sub-ranges and values therebetween. In some embodiments, the surfactant is in an amount ranging from about 1% wt to about 3% wt, or about 2% wt.

In some embodiments of the pharmaceutical compositions disclosed herein, the pharmaceutical composition is a topical formulation. In some embodiments, the topical formulation comprises porfimer sodium and when applied to the skin, provides at least 0.01% of the porfimer sodium in the epidermal-to-dermal layer of the subject. In some embodiments at least 0.03% of the porfimer sodium is retained. In some embodiments, at least 0.04% of the porfimer sodium is retained.

In some embodiments of the pharmaceutical compositions disclosed herein, the topical formulation is applied under the following conditions as described in Example 7. In some embodiments at least 0.03% of the porfimer sodium is retained in the epidermis. In some embodiments, at least 0.04% of the porfimer sodium is retained in the epidermis in conditions as described in Example 7.

In some embodiments, the present disclosure provides a pharmaceutical topical formulation containing porfimer sodium and one or more pharmaceutical acceptable excipients, wherein when applied to the skin to a subject, provides at least 0.01% of the porfimer in the epidermal-to-dermal layer of the subject. In some embodiments, the present disclosure provides a pharmaceutical topical formulation containing porfimer sodium and one or more pharmaceutical acceptable excipients, wherein when applied to the skin of a subject, provides at least 0.03% of the porfimer in the epidermal-to-dermal layer of the subject. In some embodiments, the pharmaceutical topical formulation when applied to the skin of a subject, provides at least 0.04% of the porfimer in the epidermal-to-dermal layer of the subject.

In one embodiment, the pharmaceutical compositions of the present invention are comprised of porfimer sodium and are stable compositions relative to the prior art. In some embodiments of the pharmaceutical topical formulations disclosed herein, the composition, after 40° C./75% RH for 4 weeks, has a total impurities less than 20% by HPLC. In some embodiments of the pharmaceutical composition of the present disclosure is a topical formulation. In some embodiments, the composition of the present disclosure, after 40° C./75% RH for 4 weeks, has a total impurities less than 20% by HPLC. In some embodiments, the composition of the present disclosure, after 40° C./75% RH for 4 weeks, has a total impurities less than 19% by HPLC. In some embodiments, the composition of the present disclosure, after 40° C./75% RH for 4 weeks, has a total impurities less than 18% by HPLC. In some embodiments, the composition of the present disclosure, after 40° C./75% RH for 4 weeks, has a total impurities less than 17% by HPLC. In some embodiments, the composition of the present disclosure, after 40° C./75% RH for 4 weeks, has a total impurities less than 16% by HPLC. In some embodiments, the composition of the present disclosure, after 40° C./75% RH for 4 weeks, has a total impurities less than 15% by HPLC. In some embodiments, the composition of the present disclosure, after 40° C./75% RH for 4 weeks, has a total impurities less than 14% by HPLC. In some embodiments, the composition of the present disclosure, after 40° C./75% RH for 4 weeks, has a total impurities less than 13% by HPLC. In some embodiments, the composition of the present disclosure, after 40° C./75% RH for 4 weeks, has a total impurities less than 12% by HPLC.

In some embodiments, the composition of the present disclosure, after 25° C./60% RH for 4 weeks, has a total impurities less than 20% by HPLC. In some embodiments, the composition of the present disclosure, after 25° C./60% RH for 4 weeks, has a total impurities less than 19% by HPLC. In some embodiments, the composition of the present disclosure, after 25° C./60% RH for 4 weeks, has a total impurities less than 18% by HPLC. In some embodiments, the composition of the present disclosure, after 25° C./60% RH for 4 weeks, has a total impurities less than 17% by HPLC. In some embodiments, the composition of the present disclosure, after 25° C./60% RH for 4 weeks, has a total impurities less than 16% by HPLC. In some embodiments, the composition of the present disclosure, after 25° C./60% RH for 4 weeks, has a total impurities less than 15% by HPLC. In some embodiments, the composition of the present disclosure, after 25° C./60% RH for 4 weeks, has a total impurities less than 14% by HPLC. In some embodiments, the composition of the present disclosure, after 25° C./60% RH for 4 weeks, has a total impurities less than 13% by HPLC. In some embodiments, the composition of the present disclosure, after 25° C./60% RH for 4 weeks, has a total impurities less than 12% by HPLC.

In some embodiments, the composition of the present disclosure, after 5° C./60% RH for 4 weeks, has a total impurities less than 20% by HPLC. In some embodiments, the composition of the present disclosure, after 5° C./60% RH for 4 weeks, has a total impurities less than 19% by HPLC. In some embodiments, the composition of the present disclosure, after 5° C./60% RH for 4 weeks, has a total impurities less than 18% by HPLC. In some embodiments, the composition of the present disclosure, after 5° C./60% RH for 4 weeks, has a total impurities less than 17% by HPLC. In some embodiments, the composition of the present disclosure, after 5° C./60% RH for 4 weeks, has a total impurities less than 16% by HPLC. In some embodiments, the composition of the present disclosure, after 5° C./60% RH for 4 weeks, has a total impurities less than 5% by HPLC. In some embodiments, the composition of the present disclosure, after 5° C./60% RH for 4 weeks, has a total impurities less than 14% by HPLC. In some embodiments, the composition of the present disclosure, after 5° C./60% RH for 4 weeks, has a total impurities less than 13% by HPLC. In some embodiments, the composition of the present disclosure, after 5° C./60% RH for 4 weeks, has a total impurities less than 12% by HPLC.

In other embodiments, the composition of the present disclosure, after 5° C./60% RH for 4 weeks, has an impurity of hematoporphyrin (HP) of less than 5%, 4%, 3%, or 2% by HPLC. In other embodiments, the composition of the present disclosure, after 25° C./60% RH for 4 weeks, has an impurity of hematoporphyrin (HP) of less than 5%, 4%, 3%, or 2% by HPLC. In other embodiments, the composition of the present disclosure, after 40° C./75% RH for 4 weeks, has an impurity of hematoporphyrin (HP) of less than 5%, 4%, 3%, or 2% by HPLC.

In other embodiments, the composition of the present disclosure, after 5° C./60% RH for 4 weeks, has an impurity of total hydroxyvinyl deuteroporphyrin (HVD) (isomers 1 and 2) of less than 10%, 9%, 8%, 7%, 6%, or 5% by HPLC. In other embodiments, the composition of the present disclosure, after 25° C./60% RH for 4 weeks, has an impurity of total hydroxyvinyl deuteroporphyrin (HVD) (isomers 1 and 2) of less than 10%, 9%, 8%, 7%, 6%, or 5% by HPLC. In other embodiments, the composition of the present disclosure, after 40° C./75% RH for 4 weeks, has an impurity of total hydroxyvinyl deuteroporphyrin (HVD) (isomers 1 and 2) of less than 10%, 9%, 8%, 7%, 6%, or 5% by HPLC.

In other embodiments, the composition of the present disclosure, after 5° C./60% RH for 4 weeks, has an impurity of protoporphyrin (PP) of less than 5%, 4%, 3%, or 2% by HPLC. In other embodiments, the composition of the present disclosure, after 25° C./60% RH for 4 weeks, has an impurity of protoporphyrin (PP) of less than 5%, 4%, 3%, or 2% by HPLC. In other embodiments, the composition of the present disclosure, after 40° C./75% RH for 4 weeks, has an impurity of protoporphyrin (PP) of less than 5%, 4%, 3%, or 2% by HPLC.

The compositions of the present disclosure can be utilized to treat a subject in need thereof. In certain embodiments, the subject is a mammal such as a human, or a non-human mammal. When administered to subject, such as a human, the composition or the compound may be administered as a pharmaceutical composition comprising, for example, a compound of the disclosure and a pharmaceutically acceptable carrier. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols and sugar alcohols, such as glycerin, sorbitol, mannitol, xylitol, erythritol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances, including salts such as sodium chloride, employed in pharmaceutical formulations. In some embodiments, when such pharmaceutical compositions are for human administration, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as lyophile for reconstitution, powder, solution, syrup, injection or the like. The composition can also be present in a solution suitable for topical administration.

In some embodiments, the pharmaceutical compositions of the present disclosure are formulated for topical administration. In some embodiments, the compositions of the present disclosure are formulated for topical administration to an area of non-intact skin. Non-intact skin can include, but is not limited to, skin and soft tissue infections, skin lesions, vesicles, chronic ulcers, cysts, blisters, bullae, open sores such as decubitus ulcers (bed sores) and other pressure sores, cellulitis sores, erysipelas lesions, wounds, burn wounds, carbuncles, furuncles, cutaneous ulcers, or other conditions where the skin is damaged, broken, cracked, breached and/or otherwise compromised. Topical formulations generally include a sterile buffer, such as a sterile PBS, water, or saline buffer, or a sterile SM buffer. A variety of buffers may be used in the context of the present disclosure and will be readily apparent to a person having ordinary skill in the art. For example, in some embodiments, suitable buffers include sodium or potassium citrate, citric acid, phosphate buffers such as sodium phosphate, boric acid, sodium bicarbonate and various mixed phosphate buffers including combinations of Na₂HPO₄, NaH₂PO₄ and KH₂PO₄. In some embodiments, sodium phosphate buffer is used. In some embodiments, sodium citrate buffer is used. Accordingly, the formulation pH may vary from about 5 to about 10. In some embodiments, the formulation pH is about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or about 10. In some embodiments, the formulation pH is about 7.4.

For topical application to the skin, the pharmaceutical compositions of the present disclosure may be combined with one or a combination of carriers for topical formulations. Carriers for topical formulations may comprise semi-solid and/or gel-like vehicles, which may include a polymer thickener, water, preservatives, active surfactants, emulsifiers, and/or a solvent or mixed solvent system. U.S. Pat. No. 5,863,560 discloses a number of different carrier combinations that can aid in the exposure of skin to a medicament, and its contents are incorporated herein by reference. The carrier may or may not involve a controlled-release formulation, e.g., as disclosed in US 2008/0260697, the contents of which are incorporated herein by reference. In some embodiments, the carrier may or may not involve a viscous formulation, e.g., a gel, e.g., as disclosed in US 2009/0191254, the contents of which are incorporated herein by reference.

In some embodiments, topical pharmaceutical compositions of the present disclosure are provided in a hermetically sealed container. The container may a vial, syringe, tube, bottle, ampoule (e.g., blow-fill-seal polypropylene ampules), sachet, or the like; and may comprise or consist of glass, plastic, or other suitable material. Ampoules, for example, generally are produced industrially from short lengths of glass tubing, shaped by heating with gas torches and gravity. Computer vision techniques often are employed, e.g., for quality control. The filling and sealing of ampoules may be done by automated machinery. Blank ampoules can be purchased from scientific glass supply houses and sealed, e.g., with a small gas torch, preferably under inert atmospheres. In some embodiments, the container also may be filled with an inert gas, in addition to the pharmaceutical composition. In some embodiments, the composition is provided in an ampoule, or other suitable container, and transferred for use to a vehicle suitable for direct contact with non-intact skin, e.g., a patch, wipe, bandage, dressing, as described below.

In some embodiments, topical administration of a pharmaceutical composition of the present disclosure comprises use of a dressing. The pharmaceutical composition of the present disclosure may be incorporated into a dressing and/or applied separately along with the use of a dressing. A dressing promotes healing by keeping a wound moist, creating a barrier against infection, and/or keeping the surrounding skin dry, and limiting the exposure of the PDT active ingredients to light. The degree of moisture in a wound is to be considered when treating topical wounds (e.g., skin and soft tissue infections). High levels of exudate warrant the choice of a moisture-absorbing material, including but not limited to alginates, foams, collagen-alginate combinations, carboxymethylcellulose materials, or gauze. Low exudate and desiccated wounds generally respond well to hydrogels. Hydrogel sheets often comprise three-dimensional networks of cross-linked hydrophilic polymers. Amorphous hydrogels are similar in composition to hydrogel sheets but lack the cross-linking. The gel also may comprise additional ingredients, such as collagens, alginate, or complex carbohydrates. Alginate dressings often comprise calcium or calcium-sodium salts of natural polysaccharides derived from brown seaweed. When the alginate material comes into contact with sodium-rich wound exudates, an ion exchange takes place, producing a hydrophilic gel. Additional dressing choices include but are not limited to films including adhesive-backing films, gels, and foams including silicone-coated foams, hydrocolloids, collagen-based dressings, absorbent polymers, and the like. Hydrocolloid dressings often comprise adhesive, absorbent, and elastomeric components. Carboxymethylcellulose, for example, is a common absorptive ingredient. Hydrofiber dressing also often comprise carboxymethylcellulose, for example, sodium carboxymethylcellulose. Foam dressings often comprise a polymer, often polyurethane, with small, open cells that are able to hold fluids. Some varieties of foam dressings have a waterproof film covering the top surface and may have an adhesive coating on the wound contact side or on the wound border. Film dressings often comprise a single thin transparent sheet of polyurethane coated on one side with an adhesive. The sheet is permeable to gases and water vapor but impermeable to wound fluids. Hydrofiber dressings often comprise sodium carboxymethylcellulose fibers. Collagen-based dressings often comprise purified collagen derived from bovine, porcine, equine, or avian sources. Collagen-based dressings are believed to aid wound healing e.g., by stimulating fibroblast production.

In some embodiments, topical administration of a pharmaceutical composition of the present disclosure comprises instillation. The pharmaceutical compositions of the present disclosure may be incorporated into an instillation and/or applied separately along with the use of an instillation. Instillation refers to administration by introduction of the fluid pharmaceutical composition gradually, e.g., drop by drop of the fluid. Typical instillation therapy instills fluid into a wound under a low positive pressure. Devices for use in instillation include, e.g., Kritter-type instillation catheters (see, e.g., Brent H. et al. 2005. Wounds 17(2):37-48). Techniques known in the art to improve instillation and distribution of the fluid include, but are not limited to, filling a wound with instillation fluid, applying porous wound fillers, and/or combining with negative pressure wound therapy.

Modes of administration described herein and/or known in the art may be used to deliver desired dosages of compositions of the disclosure and in accordance with suitable dosage regimens. Dosages and dosage regimens may vary depending on the particular formulation, route of administration, condition being treated, and other factors. Animal experiments may provide reliable guidance for the determination of effective doses in human therapy, e.g., as within the skill of the ordinary physician. Interspecies scaling of effective doses can be performed by one of ordinary skill in the art following the principles described, e.g., by Mordenti, J. et al. “The use of interspecies scaling in toxicokinetics” in Toxicokinetics and New Drug Development, Yacobi et al., Eds., Pergamon Press, New York 1989, pp 42-96.

The formulations may conveniently be presented in unit dosage form and can be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect.

Compositions can also be formulated so as to provide slow or controlled release of the active ingredient therein using, for example, hydroxypropylmethyl cellulose in varying proportions to provide the desired release profile, other polymer matrices, liposomes, porphysomes, nanoparticles and/or microspheres. They can be sterilized by, for example, filtration through a bacteria-retaining filter, or by incorporating sterilizing agents in the form of sterile solid compositions that can be dissolved in sterile water, or some other sterile injectable medium immediately before use.

Liquid dosage forms useful for topical administration include pharmaceutically acceptable emulsions, lyophiles for reconstitution, microemulsions, solutions, suspensions, gels, syrups and elixirs. In addition to the active ingredient, the liquid dosage forms can contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (such as cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the topical compositions can also include adjuvants such as wetting agents, emulsifying and suspending agents, and preservative agents.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound can be mixed under sterile conditions with a pharmaceutically acceptable carrier, and with any preservatives or buffers that can be required. The ointments, pastes, creams and gels can contain, in addition to an active compound, one or more excipients or carriers, such as animal and vegetable fats, oils, waxes, paraffins, starch, tragacanth, cellulose derivatives, polyethylene glycols, silicones, bentonites, silicic acid, talc, polymers, salts, and zinc oxide, or mixtures thereof. In some embodiments, the PS composition is in the form of an aqueous solution. In some embodiments, the excipient comprises a salt selected from sodium chloride. In some embodiments, the excipient comprises sodium chloride.

In some cases, in order to prolong the effect of a drug, it is desirable to slow the absorption of the drug. This can be accomplished by the use of a liquid suspension of crystalline or amorphous material having poor water solubility. The rate of absorption of the drug then depends upon its rate of dissolution, which, in turn, can depend upon crystal size and crystalline form. Alternatively, delayed absorption of a parenterally administered drug form is accomplished by dissolving or suspending the drug in an oil vehicle.

Actual dosage levels of the active ingredients in the pharmaceutical compositions can be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect, or the frequency in which the therapy is delivered to the patient. It is generally understood that the effective amount of the compound will vary according to the weight, sex, age, and medical history of the subject. Other factors which influence the effective amount can include, but are not limited to, the severity of the subject's condition, the disorder being treated, the stability of the compound, and, if desired, another type of therapeutic agent being administered with the compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable dose of an active compound used in the compositions and methods of the disclosure will be that amount of the compound that is the lowest dose effective to produce a therapeutic effect. Such an effective dose will generally depend upon the factors described above.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like. In some embodiments, the antioxidant is phenoxyethanol.

In some embodiments of the pharmaceutical compositions disclosed herein, the antioxidant is in an amount ranging from 0.01% wt to about 5 wt, including about 0.01%, about 0.05%, about 0.1% wt, about 0.2% wt, about 0.3% wt, about 0.4% wt, about 0.5% wt, about 0.6% wt, about 0.7% wt, about 0.8% wt, about 0.9% wt, about 1% wt, about 1.1% wt, about 1.2% wt, about 1.3% wt, about 1.4% wt, about 1.5% wt, about 1.6% wt, about 1.7% wt, about 1.8% wt, about 1.9% wt, about 2.0% wt, about 2.1% wt, about 2.2% wt, about 2.3% wt, about 2.4% wt, about 2.5% wt, about 2.6% wt, about 2.7% wt, about 2.8% wt, about 2.9% wt, about 3.0% wt, about 3.1% wt, about 3.2% wt, about 3.3% wt, about 3.4% wt, about 3.5% wt, about 3.6% wt, about 3.7% wt, about 3.8% wt, about 3.9% wt, about 4.0% wt, about 4.2% wt, about 4.4% wt, about 4.6% wt, about 4.8% wt, to about 5.0% wt, including all sub-ranges and values therebetween. In some embodiments, the antioxidant is in an amount ranging from 0.5% wt to about 1.5% wt, or about 1.0% wt.

The topical mode of delivery may include a smear (e.g., finger swipe or with an applicator), a spray, a foam, a bandage, a time-release patch, a liquid-absorbed wipe, and combinations thereof. In some embodiments, the compositions of the present disclosure are provided, either directly or in a carrier(s), in a patch, wipe, bandage, dressing, or other vehicle suitable for direct contact with the skin, in particular, a SSTI.

Without wishing to be bound by theory, it is believed that this success is due to the composition in combination with the photodynamic agent, porfimer sodium. The composition is able to stabilize the porfimer sodium. Further, inclusion of skin penetrants and other solvents allow the controlled release and permeation of porfimer sodium. Additionally, drying of the composition on the surface of the tissue is remedied by selection of particular compositions, especially those including a humectant. Further, selection of the appropriate viscosity of composition allows effective skin contact that enhances spreading which keeping the active agent localized to the infection site.

### Methods of Use

In some embodiments, the present disclosure provides methods for treating in a subject, damaged skin, such as skin damaged caused by sun exposure comprising administering to a subject in need thereof a therapeutically effective amount of a composition containing porfimer sodium, wherein the composition is applied to the skin, such as the sun exposed skin. In a specific embodiment, the damaged skin that may be treated can include chronically sun exposed skin, wrinkled skin, pigment spots, liver spots, actinic keratosis, seborrheic keratosis photo-damaged skin, acne, warts, and/or psoriasis and may be treated with a pharmaceutical composition containing porfimer sodium, wherein the composition is applied to the skin, such as the sun exposed skin.

In some embodiments, the present disclosure provides methods for treating damaged skin caused by sun exposure in a subject, comprising administering to a subject in need thereof any pharmaceutical composition or formulation of the present disclosure, wherein the composition is applied to the damaged skin (e.g., applied to the sun-exposed skin); and light is applied to the damaged skin. In some embodiment, the light ranges from about 380 nm to about 850 nm in wavelength. In some embodiments, the light ranges from about 400 nm to about 700 nm in wavelength. In some embodiments, the light is about 630 nm in wavelength.

In some embodiments, the present disclosure provides a method for treating chronically sun exposed skin, wrinkling, pigment spots, liver spots, actinic keratosis, seborrheic keratosis, photo-damaged skin, acne, warts, and psoriasis, comprising administering to a subject in need thereof a therapeutically effective amount of a composition a formulation of the present disclosure, wherein the composition or formulation is applied to the sun damaged skin; and light is applied to the damaged skin. In some embodiments, the light ranges from about 380 nm to about 850 nm in wavelength. In some embodiments, the light ranges from about 400 nm to about 700 nm in wavelength. In some embodiments, the light is about 630 nm in wavelength. In some embodiments, the treatment is for actinic keratosis. In some embodiments, the treatment is for seborrheic keratosis.

In some embodiments, the methods for the photodynamic treatment include treating damaged skin due to sun exposure, or treating chronically sun exposed skin, wrinkling, pigment spots, liver spots, actinic keratosis, seborrheic keratosis, photo-damaged skin, acne, warts, and psoriasis, without or the minimization of adverse reactions or side effects due to photodynamic treatment. In a specific embodiment, present disclosure provides a method for treating actinic keratosis comprising administering to a subject in need thereof a therapeutically effective amount of a composition a formulation of the present disclosure, wherein the composition or formulation is applied to the s damaged skin; and light is applied to the damaged skin, with a minimalization of adverse side effects, such as the minimization of pain, edema, pruritis and/or erythema. In a specific embodiment, the photodynamic agent is porfimer sodium. In another specific embodiment, the adverse side effects are minimalized more than other photodynamic treatment agents when applied topically to a subject. Indeed, one of the most significant drawbacks of the topical use of photodynamic agents is significant pain during light application. For example, the topical application of both 5-ALA and methyl-ALA are known to cause specific pain during light application, likely due to the fact that the drug localizes in hair follicles, nerve endings, epidermal cells and sebaceous glands. This requires novel methods to reduce pain during therapy in this field.

Accordingly, embodiments of the present invention include a method for treating damaged skin caused by sun exposure in a subject, comprising administering to the subject in need thereof any pharmaceutical composition or formulation described herein, wherein the composition is applied to the sun exposed skin; and light is applied to the sun exposed skin, wherein there is a minimalization of adverse side effects, such as the minimization of pain, edema, pruritis and/or erythema. In a specific embodiment, the pharmaceutical composition comprises a photosensitizer and one or more gelling agents. In a specific embodiment, the photosensitizer is porfimer sodium (Photofrin®).

In another specific embodiment of the methods herein, the sun exposed skin to be treated with no or minimal adverse side effects is selected from one or more of the group consisting of chronically sun exposed skin, wrinkling, pigment spots, liver spots, actinic keratosis, photo-damaged skin, acne, warts, and psoriasis. In a specific embodiment, the present invention includes a method for treating damaged skin caused by sun exposure in a subject, comprising administering to the subject in need thereof any pharmaceutical composition or formulation described herein comprising porfimer sodium, wherein the composition is applied to the sun exposed skin; and light is applied to the sun exposed skin. In a specific embodiment, the subject does not suffer from pain when the light is applied to the sun exposed skin. In another embodiment, the subject does not suffer from pain per the visual analog scale of pain when the light is applied to the sun exposed skin. See, e.g., Bodian C A, Freedman G, Hossain S, Eisenkraft J B, Beilin Y. The Visual Analog Scale for Pain. Anesthesiology 2001: 95:1356-61. In a specific embodiment, the subject suffers from a level of pain of 10, 9, 8, 7, 6, 5, 4, 3, 2, 1 or less per the visual analog scale of pain when the light is applied to the sun exposed skin. In a specific embodiment, the subject suffers from a level of pain of 5, 4, 3, 2, 1 or less per the visual analog scale of pain when the light is applied to the sun exposed skin. In a specific embodiment, the subject suffers from a level of pain of 3, 2, 1 or less per the visual analog scale of pain when the light is applied to the sun exposed skin. In a specific embodiment, the subject suffers from a level of pain of 2, 1 or less per the visual analog scale of pain when the light is applied to the sun exposed skin.

In another aspect, the present disclosure also provides methods for treating a topical skin infection, comprising administering to a subject in need thereof a therapeutically effective amount of a composition containing porfimer sodium, wherein the composition is applied to the skin infection (e.g., applied to the surface of the infection). In some embodiments, the skin infection is a skin and soft tissue infection (SSTI), the acute bacterial skin and skin structure infections (ABSSSI), both complicated and uncomplicated.

In some embodiments, the present disclosure provides methods for treating an infected area, comprising administering to a subject in need thereof any pharmaceutical composition or formulation of the present disclosure, wherein the composition is applied to the infected area (e.g., applied to the surface of the wound); and light is applied to the infected area. In some embodiment, the light ranges from about 380 nm to about 850 nm in wavelength. In some embodiments, the light ranges from about 400 nm to about 700 nm in wavelength. In some embodiments, the light is about 630 nm in wavelength. In some embodiments, the present disclosure provides a method for treating a microbial infection, comprising administering to a subject in need thereof a therapeutically effective amount of a composition a formulation of the present disclosure, wherein the composition or formulation is applied to the infection; and light is applied to the infected area. In some embodiments, the light ranges from about 380 nm to about 850 nm in wavelength. In some embodiments, the light ranges from about 400 nm to about 700 nm in wavelength. In some embodiments, the light is about 630 nm in wavelength. In some embodiments, the infected area is a wound. In a specific embodiment, the wound is a topical wound. In some embodiments, the area or topical wound is infected by one or more bacterial and/or fungal pathogens. In some embodiments, the area or topical wound is infected by antibiotic resistant bacterial pathogens.

Topical treatment provides the advantages of avoiding systemic adverse effects, providing increased target site concentration, and allowing the use of agents not available for systemic therapy, and avoiding the microbial resistance mechanisms associated with systemic therapies such as, but not limited to, biofilms. In some embodiments, mechanical debridement may be used to improve topical treatment because it reduces the bioburden of bacteria present and also opens a time-dependent therapeutic window for topical antimicrobial therapy (TAT) (Wolcott R D, et al. 2010. J Wound Care 19:320-328).

In some embodiments, the infected area is infected with one or more Gram positive bacteria. In some embodiments, the infected area is infected with one or more Gram negative bacteria. In some embodiments, the infected area is infected with one or more fungal pathogens. In some embodiments, the infected area is infected with Gram positive bacteria Staphylococcus aureus(+), Staphylococcus aureus MRSA(+), Peptostreptococcus anaerobius(+), Propionibacterium acnes(+) (now referred to as Cutibacterium acnes), Bacillus thuringiensis(+), Bacillus atrophaeus(+), Streptococcus mutans(+), Streptococcus pneumoniae(+), or with Gram negative bacteria Prevotella(−), Porphyromonas gingivalis(−), Salmonella enterica(−), Escherichia coli(−), Yersinia intermedia(−), Acinetobacter baumannii(−), Neisseria gonorrhea(−), Haemophilus influenza(−), Fusobacterium nucleatum(−), or Moraxella. catarrhalis(−). In some embodiments the infected area is infected with Candida spp., Candida albicans, Candida glabrata, Candida parasilosis, Candida krusei, Candida tropicalis, and/or Candida. guilliermondi. In some embodiments, the infected area is infected with one or more of the following Gram positive bacterial pathogens: Staphylococcus aureus(+), Staphylococcus aureus MRSA(+), Peptostreptococcus anaerobius(+), Propionibacterium acnes(+) (now referred to as Cutibacterium acnes), Bacillus thuringiensis(+), Bacillus atrophaeus(+), Streptococcus mutans(+), Streptococcus pneumoniae(+), or with Gram negative bacteria Prevotella(−), Porphyromonas gingivalis(−), Salmonella enterica(−), Escherichia coli(−), Yersinia intermedia(−), Acinetobacter baumannii(−), Neisseria gonorrhea(−), Haemophilus influenza(−), Fusobacterium nucleatum(−), or Moraxella. catarrhalis(−). In some embodiments, the infected area is infected with one or more of the following fungal pathogens: Candida, C. albicans, C. glabrata, C. parasilosis, C. krusei, C. tropicalis, C. guilliermondi. In some embodiments, the infected area is a wound. In a specific embodiment, the wound is a topical wound.

In some embodiments, the porfimer sodium compositions of the present disclosure have activity against bacterial flora found in chronic sinusitis in adults bacterial sinusitis including: Streptococcus pneumonia (Gram positive facultative anaerobe), Moraxella catarrhalis (Gram-negative, aerobe), Staphylococcus aureus (Gram-positive, facultative anaerobic), Methicillin-resistant Staphylococcus aureus (MRSA) (Gram positive facultative anaerobe), Prevotella spp. (Gram-negative, obligate anaerobe), Peptostreptococcus spp. (Gram-positive, anaerobic), Fusobacterium nucleatum (Gram-negative, anaerobic), Porphyromonas gingivalis (Gram-negative, anaerobic), S. pneumoniae (Gram positive facultative anaerobe) and Propionibacterium acnes (Gram-positive, anaerobic).

Biofilms of S. aureus and other bacteria that are present in infections like SSTIs increase the difficulty of successful infection management and reduction. Combinations of SSTI-relevant bacteria forming multispecies biofilms containing e.g., S. aureus have demonstrated greater resistance, virulence and pathogenicity than comparable single-species biofilms. The presence of such complex biofilms in SSTI patients is considered to be largely responsible for the chronic, persistent nature of these infections.

In some embodiments, the bacterial pathogen exhibits resistance to one or more antibiotics. Of particular concern are the methicillin-resistant Staphylococcus aureus strains (MRSA). MRSA remained an uncommon occurrence in hospital setting until the 1990's, when there was an explosion in MRSA prevalence in hospitals. MRSA now is considered endemic to hospitals, especially in the UK (Johnson A P et al. 2001 J. Antimicrobial Chemotherapy 48(1): 143-144). Moreover, MRSA presents a new threat in diabetic foot infections (Retrieved Jan. 17, 2009, from CDC: Centers for Disease Control and Prevention Web site).

The porfimer sodium compositions of the present disclosure have activity against a plurality of bacterial and fungal strains. In some embodiments, the porfimer sodium compositions have activity in-vitro and in-vivo against a plurality of strains including but not limited to Gram positive bacteria Staphylococcus aureus(+), Staphylococcus aureus MRSA(+), Peptostreptococcus anaerobius(+), Propionibacterium acnes(+) (now referred to as Cutibacterium acnes), Bacillus thuringiensis(+), Bacillus atrophaeus(+), Streptococcus mutans(+), Streptococcus pneumoniae(+), or with Gram negative bacteria Prevotella(−), Porphyromonas gingivalis(−), Salmonella enterica(−), Escherichia coli(−), Yersinia intermedia(−), Acinetobacter baumannii(−), Neisseria gonorrhea(−), Haemophilus influenza(−), Fusobacterium nucleatum(−), or Moraxella. catarrhalis(−), or one or more of the following fungal pathogens: Candida, C. albicans, C. glabrata, C. parasilosis, C. krusei, C. tropicalis, C. guilliermondi.

Accordingly, some embodiments of the present disclosure provide methods of treating and/or preventing infections and wounds associated with Gram positive bacteria Staphylococcus aureus(+), Staphylococcus aureus MRSA(+), Peptostreptococcus anaerobius(+), Propionibacterium acnes(+) (now referred to as Cutibacterium acnes), Bacillus thuringiensis(+), Bacillus atrophaeus(+), Streptococcus mutans(+), Streptococcus pneumoniae(+), or with Gram negative bacteria Prevotella(−), Porphyromonas gingivalis(−), Salmonella enterica(−), Escherichia coli(−), Yersinia intermedia(−), Acinetobacter baumannii(−), Neisseria gonorrhea(−), Haemophilus influenza(−), Fusobacterium nucleatum(−), or Moraxella. catarrhalis(−) or Candida spp., Candida albicans, Candida glabrata, Candida parasilosis, Candida krusei, Candida tropicalis, and/or Candida. Guilliermondi.

In some specific embodiments, the methods of treating and/or preventing infections or wounds are associated with Staphylococcus aureus, Staphylococcus aureus MRSA, P. anaerobius, P. acnes, B. thuringiensis, Bacillus. atrophaeus, S. mutans, S. pneumoniae, Prevotella, P. gingivalis, S. enterica, E. coli, Y. intermedia, A. baumannii, N. gonorrhoea, H. influenza, E. nucleatum, M catarrhalis or one or more of the following fungal pathogens: Candida spp., C. albicans, C. glabrata, C. parasilosis, C. krusei, C. tropicalis, C. guilliermondi in both humans and animals using the porfimer sodium compositions. In other aspects, the present disclosure provides methods of treating and/or preventing infections associated with related species or strains of these bacteria. In some embodiments, the bacterial infection is a SSTI. Staphylococcus aureus, Staphylococcus aureus MRSA, P. anaerobius, P. acnes, B. thuringiensis, B. atrophaeus, S. mutans, S. pneumoniae, Prevotella, P. gingivalis, S. enterica, E. coli, Y. intermedia, A. baumannii, N. gonorrhoea, H. influenza, F. nucleatum, M. catarrhalis are responsible for many severe opportunistic infections particularly in individuals with SSTIs.

In some embodiments, the pharmaceutical compositions of the present disclosure are contemplated for treating and/or preventing a wound, associated with Staphylococcus aureus, Staphylococcus aureus MRSA, P. anaerobius, P. acnes, B. thuringiensis, B. atrophaeus, S. mutans, S. pneumoniae, Prevotella spp., P. gingivalis, S. enterica, E. coli, Y. intermedia, A. baumannii, N. gonorrhoea, H. influenza, F. nucleatum, M. catarrhalis. In some embodiments, the wound is infected with one or more of the following fungal pathogens: Candida spp., C. albicans, C. glabrata, C. parasilosis, C. krusei, C. tropicalis, C. guilliermondi or associated with other species or strains of bacteria, including, but not limited to, infected area such as infections of the skin, infections in and around wounds, chronic ulcers, ulcers associated with burn wounds, post-operative infections, infections associated with catheters and surgical drains, and infections of the blood.

PDT using porfimer sodium compositions of the present disclosure find use as an effective noninvasive or minimally invasive treatment for oral, head & neck, superficial and/or subcutaneous infections (e.g., bacterial and/or fungal infections) including, but not limited to, infections resulting from sinusitis, wounds, open fractures, surgical implants, and surgical procedures. In some embodiments, the compositions find use in treating and/or preventing bacterial infections associated with areas of non-intact skin, including but not limited to, infections associated with cutaneous ulcers, skin lesions, vesicles, cysts, blisters, bullae, open sores such as decubitus ulcers (bed sores) and other pressure sores, chronic ulcers, cellulitis, impetigo and sores associated therewith, erysipelas and lesions associated therewith, wounds, burns and wounds associated therewith, carbuncles, furuncles or other conditions where the skin is damaged, cracked, broken, breached, and/or otherwise compromised.

In any of the embodiments described herein, the porfimer sodium compositions may be used to treat an infection (e.g. SSTI, ABSSSI) of one or more of the following fungal pathogens: Candida, spp., C. albicans, C. glabrata, C. parasilosis, C. krusei, C. tropicalis, C. guilliermondi.

In some embodiments of the methods for treating an infected area, after administration of the porfimer sodium composition, one or more of the following occurs: (i) reducing and or dispersing a microbial (e.g., bacterial and/or fungal) biofilm, (ii) impairing growth or formation of a microbial (e.g., bacterial and/or fungal) biofilm, and (iii) preventing reformation or spread of a microbial (e.g., bacterial and/or fungal) biofilm. In some embodiments, the porfimer sodium composition treats, manages, and/or lessens the severity of a skin and soft tissue bacterial or fungal infection by one or both of: (i) prevention of the infection by the bacterial or fungal pathogen; and/or (ii) reduction of the bacterial or fungal pathogen. In some embodiments, the porfimer sodium composition treats, manages or lessens the severity of the infection by one or more of: (i) prevention of elaboration or secretion of exotoxins from the bacterial or fungal pathogen; (ii) inhibition of cell viability or cell growth of planktonic cells of the bacterial or fungal pathogen; (iii) inhibition of biofilm formation by the bacterial or fungal pathogen; (iv) inhibition of biofilm or microbial pathogen invasiveness to underlying tissues (e.g. subcutaneous tissue); (v) inhibition of biofilm or microbial pathogen pathogenicity to underlying tissues (e.g. subcutaneous tissue); (vi) inhibition of biofilm viability or biofilm growth of biofilm-forming cells of the bacterial or fungal pathogen; and/or (vii) prevents the reformation of biofilm after debridement. In some embodiments, the subject experiences two or more of the recited outcomes. In some embodiments, the subject experiences three or more of the recited outcomes. In some embodiments, the subject experiences four or more of the recited outcomes. In some embodiments, the subject experiences all of the recited outcomes.

In some embodiments of the methods of the present disclosure, after administration of the composition or formulation, one or more of the following occurs: (i) reducing and or dispersing a bacterial and/or fungal biofilm, (ii) impairing growth or formation of a bacterial and/or fungal biofilm, and (iii) preventing reformation or spread of a bacterial and/or fungal biofilm. In some embodiments, the subject experiences at least one of the recited outcomes. In some embodiments, the subject experiences at least two of the recited outcomes. In some embodiments, the subject experiences all of the recited outcomes. In some embodiments of the methods for treating an infected area, the subject experiences one or more of the following outcomes following the completion of dosing: less reinfection/relapse; resolution or improvement in signs and/or symptoms of infection that include redness, swelling, induration, exudate, pain, warmth (at site of infection) or fever; improved quality of life; eradication of insulting pathogens and/or biofilm; reduced need for concurrent systemic antibiotics.

In some embodiments, the methods for the photodynamic treatment include treating an infection without or the minimization of adverse reactions or side effects due to photodynamic treatment. In a specific embodiment, when the composition and then light is applied to the infection, skin or wound of the subject, there is a minimalization of adverse side effects, such as the minimization of pain, edema, pruritis and/or erythema. In a specific embodiment, the photodynamic agent is porfimer sodium. In another specific embodiment, the adverse side effects are minimalized more than other photodynamic treatment agents when applied topically to a subject. Indeed, one of the most significant drawbacks of the topical use of photodynamic agents is significant pain during light application. For example, the topical application of both 5-ALA and methyl-ALA are known to cause specific pain during light application, likely due to the fact that the drug localizes in hair follicles, nerve endings, epidermal cells and sebaceous glands. This requires novel methods to reduce pain during therapy in this field.

Accordingly, embodiments of the present invention include a method for treating wounds in a subject, comprising administering to the subject in need thereof any pharmaceutical composition or formulation described herein, wherein the composition is applied to the subject; and light is applied to the subject, wherein there is a minimalization of adverse side effects, such as the minimization of pain, edema, pruritis and/or erythema. In a specific embodiment, the pharmaceutical composition comprises a photosensitizer and one or more gelling agents. In a specific embodiment, the photosensitizer is porfimer sodium (Photofrin®).

In a specific embodiment, the subject does not suffer from pain when the light is applied to the infection, wound or skin of the subject. In another embodiment, the subject does not suffer from pain per the visual analog scale of pain when the light is applied to the infection, wound or skin of the subject. See, e.g., Bodian C A, Freedman G, Hossain S, Eisenkraft J B, Beilin Y. The Visual Analog Scale for Pain. Anesthesiology 2001: 95:1356-61. In a specific embodiment, the subject suffers from a level of pain of 10, 9, 8, 7, 6, 5, 4, 3, 2, 1 or less per the visual analog scale of pain when the light is applied to the subject. In a specific embodiment, the subject suffers from a level of pain of 5, 4, 3, 2, 1 or less per the visual analog scale of pain when the light is applied to the subject. In a specific embodiment, the subject suffers from a level of pain of 3, 2, 1 or less per the visual analog scale of pain when the light is applied to the subject. In a specific embodiment, the subject suffers from a level of pain of 2, 1 or less per the visual analog scale of pain when the light is applied to the subject.

In a specific embodiment of the methods herein, the pharmaceutical composition is applied to the infection and/or skin of the subject for about 1, 2, 3, 4, 5, 6, 7, 8, 9, 10, 11, 12, 12, 14, 14, 16, 17, 18, 19, 20, 21, 22, 23, 24, 25, 26, 27, 28, 29, 30, 35, 40, 45, 50, 55, 60, or 120 minutes. In another embodiment, the light is applied to the subject for about 1, 2, 3, 4, 5, 6, 7, 8, 9, 10, 11, 12, 12, 14, 14, 16, 17, 18, 19, 20, 21, 22, 23, 24, 25, 26, 27, 28, 29, 30, 35, 40, 45, 50, 55, 60, or 120 minutes.

In a specific embodiment, the composition is applied to the subject for about 15 to about 60 minutes, and light is applied for about 5 to about 25 minutes. In a specific embodiment, the composition is applied to the subject for about 15 to about 30 minutes, and light is applied for about 5 to about 25 minutes. In another embodiment, the subject does not develop erythematous lesions. In another embodiment, the subject does not develop erythematous lesions higher than 1 or 2 per the erythema scale from treatment on the subject,

In a specific embodiment, the composition is applied to the subject for about 15 to 60 minutes, and light is applied for 5 minutes. In a specific embodiment, the composition is applied to the subject for about 15 to 30 minutes, and light is applied for 5 minutes. In a specific embodiment, the light is applied for 5-25 minutes. In a specific embodiment, the composition is applied to the subject as provided in any one of Examples 7-11 provided herein. In another embodiment, the subject does not develop edema and/or pruritis from treatment on the sun exposed or damaged skin. In another embodiment, the subject does not develop erythematous lesions from treatment on the damaged or sun exposed skin. In another embodiment, the subject does not develop erythematous lesions higher than an increase of 1 or 2 per the erythema scale from treatment on the sun exposed skin.

In some embodiments of the methods disclosed herein, the porfimer sodium composition is administered three times per day, two times per day, once daily, every other day, once every three days, three times per week, once every week, once every other week, once every month, or once every other month. In some embodiments, the composition is administered once daily or three times per week. In some embodiments, the subject is administered multiple doses of the porfimer sodium composition daily or weekly for a length of time ranging from about one week to about 12 weeks. In some embodiments, the subject is administered multiple doses of the porfimer sodium composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the porfimer sodium composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments of the methods disclosed herein, the porfimer sodium composition is administered three times per day, two times per day, once daily, every other day, once every three days, three times per week, once every week, once every other week, once every month, or once every other month. In some embodiments, the composition is administered once daily or three times per week. In some embodiments, the subject is administered multiple doses of the porfimer sodium composition daily or weekly for a length of time ranging from about one week to about 12 weeks. In some embodiments, the subject is administered multiple doses of the porfimer sodium composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the porfimer sodium composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

### EMBODIMENTS

1. A pharmaceutical composition comprising a photosensitizer and one or more gelling agents.
2. The pharmaceutical composition of embodiment 1, wherein the photosensitizer is selected from one or more of the group consisting of porphyrins, chlorins (HPPH; NPe6; Temoporfrin (Foscan), mTHPC)), and porphysomes as in: pyropheophorbide nanovesicles including, bacteriochlorophyll porphysomes, zinc pyropheophorbide porphysomes and pyropheophyorbide porphysomes, and chlorin-like compounds (benzoporphyrin; Verteporfin, bacteriochlorins and phthalocyanines, purpurins (tin ethyl etiopurpurin); Metalloporphyrins (Texaphyrins); Pheophorbides (TOOKAD); Protoporphyrins (Levulan, Metvix, 5-ALA (PpIX)) and nonporphyrin based photosensitizers including phenothiazinium salts such as Methylene Blue, Toluidine Blue, Nile Blue, Cyanines, hypericin and Chalcogenpyrilium dyes; PPA904; benzophenothiazinium dye EtNBS; the xanthene class of fluorescent dyes that includes fluorescein and Rose Bengal; Fullerenes (C60 fullerene coupled to polar diserinol groups or quaternary pyrrolidinium groups); Squaraogenines, BODIPY (boron-dipyrromethene) dye, Phenalenones; Hypericin, Hypocrellin, Riboflavin, Curcumin, Titanium dioxide and porfimer sodium (Photofrin®).
3. The pharmaceutical composition of embodiment 1, wherein the photosensitizer is porfimer sodium.
4. The pharmaceutical composition of embodiment 3, wherein the porfimer sodium is in an amount ranging from about 0.01% wt to about 1.0% wt; or about 0.05% wt to about 0.7% wt; or about 0.1% wt to about 0.5% wt; or about 0.15% wt to about 0.3% wt.
5. The pharmaceutical composition of embodiment 3, wherein the porfimer sodium is an amount ranging from 0.01% wt to 1.0% wt; or 0.05% wt to 0.7% wt; or 0.1% wt to 0.5% wt; or 0.15% wt to 0.3% wt.
6. The pharmaceutical composition of any one of embodiments 1-5, wherein the gelling agent is selected from one or more of the group consisting of a Carbopol polymer, carbomer, crosslinked, polyacrylic acid, lecithin such as Lecithin-PLO, hydroxyethyl cellulose, hydroxypropyl cellulose, and hydroxypropyl methylcellulose
7. The pharmaceutical composition of embodiment 6, wherein at least one gelling agent is a carbomer
8. The pharmaceutical composition of embodiment 7, wherein the gelling agent is a polymer of acrylic acid cross-linked with polyalkenyl ethers or divinyl glycol.
9. The pharmaceutical composition of embodiment 7, wherein the carbomer is a Carbopol polymer Carbopol® 71G, Carbopol® 971P, Carbopol® 974P, Carbopol® 980, Carbopol® 981, Carbopol® 5984, Carbopol® 934, Carbopol® 934P, Carbopol® 940, Carbopol® 941, and/or Carbopol® 1342.
10. The pharmaceutical composition of any one of embodiments 1-9, wherein the gelling agent is in an amount ranging from about 0.5% wt to about 3.0% wt, or from about 0.7% wt to about 2.0% wt, or from about 1.0% wt to about 1.75% wt.
11. The pharmaceutical composition of any one of embodiments 1-9, wherein the gelling agent is in an amount ranging from 0.5% wt to 3.0% wt, or from 0.7% wt to 2.0% wt, or from 1.0% wt to 1.75% wt.
12. The pharmaceutical composition of any one of embodiments 1-10, wherein the pharmaceutical composition further comprises one or more permeation enhancers.
13. The pharmaceutical composition of embodiment 7, wherein the permeation enhancer is selected from one or more of the group consisting of propylene glycol SR, polyethylene glycol 400 SR, polyethylene glycol 300 LA, diethylene glycol monoethyl ether, dimethyl sulfoxide (DMSO), and Polysorbate 80 SR.
14. The pharmaceutical composition of embodiment 13, wherein the permeation enhancer is selected from one propylene glycol SR and diethylene glycol monoethyl ether.
15. The pharmaceutical composition of any one of embodiments 12-14 wherein the permeation enhancer is in an amount ranging from about 1.0% to about 60%, or about 5% to about 30%.
16. The pharmaceutical composition of any one of embodiments 12-15, wherein the pharmaceutical composition comprises a photosensitizer, a gelling agent, one or more permeation enhancers, a humectant, a solubilizer, and a preservative.
17. The pharmaceutical composition of embodiment 16, wherein:
i) the humectant is in an amount ranging from about 10% wt to about 20% wt, or about 15% wt;
ii) the solubilizer is in an amount ranging from 1.0% wt to about 5% wt, or about 1% to about 3%, or about 2%; and/or
iii) the preservative is in an amount ranging from 0.5% wt to about 5% wt, or about 0.5% wt to about 2% wt, or about 1% wt to about 3% wt.
   18. The pharmaceutical composition of any one of embodiments 1-17, wherein the pharmaceutical composition is a topical formulation.
   19. The pharmaceutical composition of embodiment 18, wherein the topical formulation comprises porfimer sodium and when applied to human skin under the conditions as provided in Example 7, provides at least 0.03% percent of the porfimer in the epidermis of the subject.
   20. The pharmaceutical composition of embodiment 18, wherein the topical formulation comprises porfimer sodium and when applied to woundless human skin of a subject, provides at least 0.03% percent of the porfimer in the epidermal-dermal layer of the subject.
   21. The pharmaceutical composition of embodiment 19 or 20, wherein the composition, after 40° C./75% RH for 4 weeks, has a total impurities less than 20% by HPLC.
   22. A pharmaceutical topical formulation comprising porfimer sodium and one or more pharmaceutical acceptable excipients, wherein when applied to the, infected area, or wound of a subject, provides at least 0.03% percent of the porfimer in the dermis of the subject.
   23. The pharmaceutical topical formulation of embodiment 19, wherein the topical formulation provides at least 0.04% percent of the porfimer in the epidermis of the subject.
   24. The pharmaceutical topical formulation of embodiment 22 or 23, wherein the composition, after 40° C./75% RH for 4 weeks, has a total impurities less than 20% by HPLC.
   25. The pharmaceutical topical formulation of any one of embodiments 22-24, wherein the pharmaceutical topical formulation further comprises a gelling agent, one or more permeation enhancers.
   26. A method for treating sun exposed skin in a subject, comprising administering to the subject in need thereof any pharmaceutical composition of any one of embodiments 1-25, wherein the composition is applied to the sun exposed skin; and light is applied to the sun exposed skin.
   27. The method of embodiment 26, wherein the light ranges from about 380 nm to about 850 nm in wavelength.
   28. The method of embodiment 27, wherein the light is about 630 nm in wavelength.
   29. The method of any one of embodiments 26-28 wherein the sun exposed skin to be treated is selected from one or more of the group consisting of chronically sun exposed skin, wrinkling, pigment spots, liver spots, actinic keratosis, seborrheic keratosis, photo-damaged skin, acne, warts, and psoriasis.
   30. The method of any one of embodiments 26-29, wherein the subject does not suffer from pain when the light is applied to the sun exposed skin.
   31. The method of embodiment 30, wherein the subject does not suffer from pain per the visual analog scale of pain when the light is applied to the sun exposed skin.
   32. The method of embodiment 31, wherein the subject suffers from a level pain of 5, 4, 3, 2, 1 or less per the visual analog scale of pain when the light is applied to the sun exposed skin.
   33. The method of any one of embodiments 26-32, wherein the composition is applied to the skin or wound of the subject for 15 to 60 minutes, and light is applied for 5-25 minutes.
   34. The method of any one of embodiments 26-33, wherein the composition is applied to the subject as provided in any one of Examples 4-14 herein.
   35. The method of any one of embodiments 26-34, wherein the subject does not develop edema or pruritis from treatment on the sun exposed skin.
   36. The method of any one of embodiments 26-35, wherein the subject does not develop erythematous lesions from treatment on the sun exposed skin.
   37. The method of any one of embodiments 26-36, wherein the subject does not develop erythematous lesions higher than 1 or 2 per the erythema scale from treatment on the sun exposed skin.
   38. The method of embodiment 37, wherein the composition is applied to the skin or wound of the subject for 15 to 60 minutes, and light is applied for 5-25 minutes.
   39. The method of embodiment 38, wherein the composition is applied to the subject as provided in any one of Examples 6-14 herein.
   40. The method of any one of claims 26-39, wherein the subject is treated for actinic keratosis and/or seborrheic keratosis.
   41. The pharmaceutical composition of any one described in Table 1 or Table 5.
   42. A method for treating sun exposed skin in a subject, comprising administering to the subject in need thereof any pharmaceutical composition of embodiment 41, wherein the composition is applied to the sun exposed skin; and light is applied to the sun exposed skin.
   43. A method for treating an infected area, comprising administering to a subject in need thereof the topical pharmaceutical composition of embodiment 41, wherein the pharmaceutical composition is applied to the infected area.
   44. The method of embodiment 42 or 43, wherein the subject is administered light at about 630 nm in wavelength applied to the infected area.
   45. The method of embodiment 43 or 44, wherein the infected area is due to a microbial infection.
   46. The method of embodiment 45, wherein the infected area is infected with with Gram positive bacteria Staphylococcus aureus(+), Staphylococcus aureus MRSA(+), Peptostreptococcus anaerobius(+), Propionibacterium acnes(+) (now referred to as Cutibacterium acnes), Bacillus thuringiensis(+), Bacillus atrophaeus(+), Streptococcus mutans(+), Streptococcus pneumoniae(+), and/or with Gram negative bacteria Prevotella(−), Porphyromonas gingivalis(−), Salmonella enterica(−), Escherichia coli(−), Yersinia intermedia(−), Acinetobacter baumannii(−), Neisseria gonorrhea(−), Haemophilus influenza(−), Fusobacterium nucleatum(−), or Moraxella. catarrhalis(−) and/or with Candida spp., Candida albicans, Candida glabrata, Candida parasilosis, Candida krusei, Candida tropicalis, and/or Candida. Guilliermondi.

### EXAMPLES

The following examples are provided to illustrate the present disclosure, and should not be construed as limiting thereof.

### Example 1: Porfimer Sodium Formulations 1-6

The application of successful in vitro work with conventional porfimer sodium solutions to in vivo studies is extremely challenging. For example, the in vivo studies described of antimicrobial photodynamic therapy with conventional FDA approved porfimer sodium preparations gave negative or mixed results in models of abraded wounds, despite promising result in vitro. Similarly, formulations containing conventional reconstituted porfimer sodium, such as mixed with thickening agent, do not improve in vivo efficacy, as poor skin permeation of the porfimer sodium was effected in saline solution. Conventional FDA approved porfimer sodium preparations have poor stability once reconstituted into solution. As such, reconstituted porfimer sodium must be used immediately, or within 24 hours, upon reconstitution. The compositions of more complex and advanced porfimer sodium (0.2% w/w) formulations Formulation 1-Formulation 6 prepared are outlined in Table 1 below.

TABLE 1
Topical Porfimer Sodium Formulations 1-6

| | | % w/w | | | | | |
|---|---|---|---|---|---|---|---|
| | | Water-based | | | | | |
| | | Formulation | Anhydrous Formulations | | | | |
| Ingredient | Functionality | 1 | 2 | 3 | 4 | 5 | 6 |
| Porfimer sodium | API | 0.2 | 0.2 | 0.2 | 0.2 | 0.2 | 0.2 |
| Phosphate | Buffer | 20.0 | | | | | |
| buffered saline | | | | | | | |
| Propylene glycol | Preservative; | 37.3 | 57.3 | 57.3 | 30.0 | 20.0 | 20.0 |
| SR | Humectant; | | | | | | |
| | Stabilizing | | | | | | |
| | Agent; | | | | | | |
| | Solubilizing | | | | | | |
| | Agent; | | | | | | |
| | Permeation | | | | | | |
| | Enhancer | | | | | | |
| Polyethylene | Solvent; | | | | | 57.8 | 20.0 |
| glycol 400 SR | Permeation | | | | | | |
| | Enhancer | | | | | | |
| Polyethylene | Solvent; | 20.0 | 20.0 | | 46.8 | | |
| glycol 300 LA | Permeation | | | | | | |
| | Enhancer | | | | | | |
| Diethylene | Solubilizing | | | 20.0 | | | 22.8 |
| glycol | Agent; | | | | | | |
| monoethyl ether | Permeation | | | | | | |
| | Enhancer | | | | | | |
| Polysorbate 80 | Emulsifier; | 2.0 | 2.0 | 2.0 | 2.0 | 2.0 | 2.0 |
| | Solubilizing | | | | | | |
| | Agent; | | | | | | |
| | Permeation | | | | | | |
| | Enhancer; | | | | | | |
| | Surfactant | | | | | | |
| Glycerin | Preservative; | 15 | 15 | 15 | 15 | 15 | 15 |
| | Emollient; | | | | | | |
| | Humectant; | | | | | | |
| | Solvent | | | | | | |
| Benzyl alcohol | Solubilizing | 2.0 | 2.0 | 2.0 | 2.0 | 2.0 | 2.0 |
| | Agent; | | | | | | |
| | Preservative | | | | | | |
| Phenoxyethanol | Preservative/ | 1.0 | 1.0 | 1.0 | 1.0 | 1.0 | 1.0 |
| | Antioxidant | | | | | | |
| Hexylene glycol | Solvent; | 1.0 | 1.0 | 1.0 | 1.0 | 1.0 | 1.0 |
| | Permeation | | | | | | |
| | Enhancer | | | | | | |
| Hydroxy propyl | Emulsifying | 1.5 | 1.5 | | | | |
| cellulose | Agent; | | | | | | |
| | Stabilizing | | | | | | |
| | Agent; Gelling | | | | | | |
| | agent | | | | | | |
| Carbomer | Emulsifying | | | 1.5 | 2.0 | | |
| | Agent; | | | | | | |
| | Stabilizer; | | | | | | |
| | Rheology | | | | | | |
| | Modifier; | | | | | | |
| | Gelling Agent | | | | | | |
| Lecithin | Emollient; | | | | | 1.0 | |
| | Emulsifying | | | | | | |
| | Agent; | | | | | | |
| | Solubilizing | | | | | | |
| | Agent | | | | | | |
| Stearyl alcohol | Bodifying agent | | | | | | 1.0 |
| Polyethylene | Solvent; | | | | | | 15.0 |
| glycol 4000 | Surfactent | | | | | | |

### Example 2: Stability and Skin Permeation Studies on Formulations 1-5

Stability Studies

The stability of porfimer sodium formulations 1 through 5 were tested. Each formulation was tested for physical and chemical stability. Physical testing includes appearance (visual description and color, of the product), pH and microscopy. Chemical testing includes, assay (% LC) and impurities, specifically looking for degradation products of porfimer sodium. Specific impurities/degradation products analyzed for detection by HPLC retention time include hematoporphyrin (HP) and hydroxyvinyl deuteroporphyrin (HVD) (isomers 1 and 2) which are hydrolysis products of porfimer sodium and protoporphyrin (PP), which is a dehydration product. The total impurities is an accumulation of these impurities.

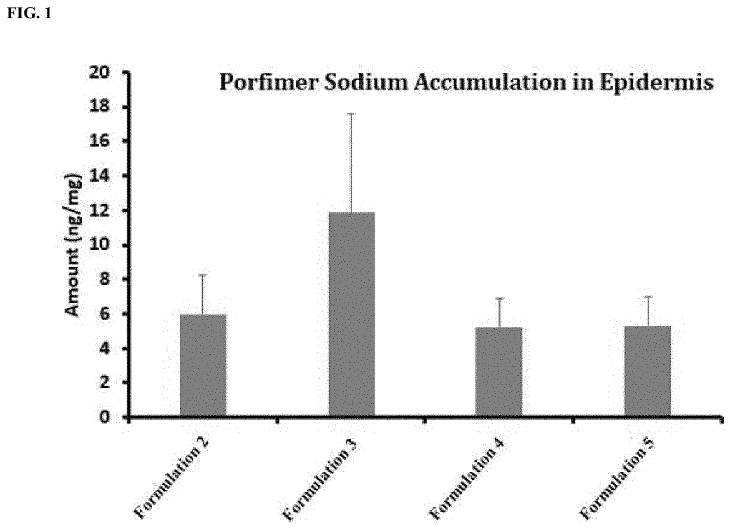

Environmental Chambers are maintained at the following condition temperature (° C.) and relative humidity (RH): Long term storage: 25±2° C./60±5% RH. Refrigerated: 5±3° C. Accelerated storage: 40±2° C./75±5% RH. The results are summarized in FIG. 1 and Table 2 below.

TABLE 2

| | | | Photofrin Sodium | | | | | |
|---|---|---|---|---|---|---|---|---|
| | | | Assay | | | | | Total impurities |
| Formulation | Timepoint | Condition | (%) | Hp | HvD1 | HvD2 | Pp | (% by HPLC) |
| 1 | T0 (Initial) | RT | 104.4 | 2.25 | 2.40 | 7.00 | 4.32 | 15.97 |
| | T2 wk | 5° C. | 109.5 | 2.75 | 2.57 | 7.69 | 4.34 | 17.35 |
| | | 25° C./60% RH | 109.5 | 3.22 | 2.88 | 8.07 | 4.56 | 18.73 |
| | | 40° C./75% RH | 104.5 | 5.52 | 4.29 | 9.19 | 4.36 | 23.36 |
| | T4 wk | 5° C./60% RH | 108.9 | 2.22 | 2.28 | 6.91 | 3.59 | 15.01 |
| | | 25° C./60% RH | 109.7 | 3.24 | 2.94 | 7.69 | 4.03 | 17.91 |
| | | 40° C./75% RH | 95.8 | 6.28 | 4.69 | 9.19 | 4.19 | 24.35 |
| 2 | T0 (Initial) | | 85.0 | 1.52 | 1.62 | 5.21 | 3.50 | 11.85 |
| | T2 wk | 5° C. | 91.8 | 1.79 | 1.75 | 5.99 | 3.80 | 13.33 |
| | | 25° C./60% RH | 81.3 | 1.82 | 1.59 | 5.44 | 3.41 | 12.26 |
| | | 40° C./75% RH | 84.1 | 2.91 | 1.75 | 5.81 | 3.33 | 13.79 |
| | T4 wk | 5° C./60% RH | 93.2 | 1.61 | 1.31 | 5.34 | 3.23 | 11.50 |
| | | 25° C./60% RH | 91.6 | 1.88 | 1.40 | 5.24 | 3.29 | 11.81 |
| | | 40° C./75% RH | 80.7 | 3.32 | 1.85 | 5.85 | 3.21 | 14.24 |
| 3 | T0 (Initial) | | 94.0 | 2.15 | 2.02 | 6.10 | 3.61 | 13.88 |
| | T2 wk | 5° C. | 100.6 | 2.55 | 2.03 | 6.56 | 3.86 | 15.00 |
| | | 25° C./60% RH | 95.2 | 2.81 | 2.00 | 6.45 | 3.74 | 15.00 |
| | | 40° C./75% RH | 89.5 | 4.89 | 2.34 | 6.94 | 3.25 | 17.41 |
| | T4 wk | 5° C./60% RH | 104.8 | 2.26 | 1.57 | 6.10 | 3.49 | 13.42 |
| | | 25° C./60% RH | 93.5 | 2.81 | 1.66 | 5.62 | 3.03 | 13.11 |
| | | 40° C./75% RH | 79.1 | 4.91 | 2.10 | 5.66 | 3.00 | 15.67 |
| 4 | T0 (Initial) | | 80.7 | 1.40 | 1.56 | 4.90 | 3.12 | 10.98 |
| | T2 wk | 5° C. | 90.4 | 1.64 | 1.66 | 5.77 | 3.53 | 12.60 |
| | | 25° C./60% RH | 84.2 | 1.70 | 1.62 | 5.38 | 3.20 | 11.90 |
| | | 40° C./75% RH | 74.3 | 2.42 | 1.66 | 5.78 | 2.57 | 12.42 |
| | T4 wk | 5° C./60% RH | 87.0 | 1.40 | 1.17 | 4.97 | 2.88 | 10.42 |
| | | 25° C./60% RH | 84.3 | 1.65 | 1.24 | 4.72 | 2.78 | 10.38 |
| | | 40° C./75% RH | 65.9 | 2.23 | 1.37 | 4.08 | 2.52 | 10.20 |
| 5 | T0 (Initial) | | 97.7 | 1.93 | 1.94 | 5.96 | 3.88 | 13.71 |
| | T2 wk | 5° C. | 101.5 | 2.22 | 2.17 | 7.02 | 4.37 | 15.78 |
| | | 25° C./60% RH | 99.9 | 2.52 | 2.11 | 6.55 | 3.97 | 15.15 |
| | | 40° C./75% RH | 105.4 | 3.03 | 1.92 | 6.77 | 4.23 | 15.95 |
| | T4 wk | 5° C. | 101.10 | 2.02 | 1.27 | 5.69 | 3.30 | 12.28 |
| | | 25° C./60% RH | 104.1 | 2.43 | 1.54 | 6.16 | 3.68 | 13.80 |
| | | 40° C./75% RH | 102.4 | 3.07 | 1.77 | 6.45 | 3.93 | 15.21 |

As shown in Table 2, formulations 3 and 5 were physically and chemically stable for 4 weeks at 5° C., 25° C./60% RH and 40° C./75% RH except for formulation 1 that shows more than 24% impurities at 40° C. This was a surprising and unexpected result, given that porfimer sodium is only stable for 24 hours when reconstituted with saline.

### Example 3: Skin Permeation Studies

A study was completed to show the ability of the formulation to deliver the drug into human skin in an in vitro setting, while retaining the API within the appropriate skin layers. The study design parameters is shown in Table 3 below.

TABLE 3
Skin Penneation Study Groups

| | Porfimer | | | Formulation |
|---|---|---|---|---|
| | Sodium | Numbers of | Repli- | Dose Volume |
| Formulations | Concentration | Donors | cates | (μL) |
| Formulation 2 | 0.2% w/w | 1 | 5 | 7.8 |
| Formulation 3 | 0.2% w/w | 1 | 5 | 7.8 |
| Formulation 4 | 0.2% w/w | 1 | 5 | 7.8 |
| Formulation 5 | 0.2% w/w | 1 | 5 | 7.8 |

In this experiment, TEWL for each diffusion cell was measured and recorded before test article applications. A range of not more than 25 g/m2/h was acceptable for this study. An automated in-line flow through diffusion cell system (PermeGear Collector FC 33, Version 3.1) was used to assess drug in the skin permeation experiment. Test articles were uniformly dispensed onto the skin tissue surface using a positive displacement pipette, set to deliver 7.8 pt. The study was performed under dark environment and closed to ambient conditions. Fractions were collected at intervals as indicated in the protocol for up to 24-hour time point. Precautions should be taken to prevent exposure of light in samples. Fractions were collected immediately capped and stored in aluminum foil at −20° C. At the end of the testing time, the surface of the tissue was cleaned under dark environment, with 1 dry cotton swab, 1 wet cotton swab in wash solvent (Hexane:Acetronitrile 1:9 v/v), and 1 dry cotton swab followed by three consecutive tape strips. The swabs and the tape strips were analyzed upon request. The washed and tape stripped skin was placed on aluminum foil with dermis side down and processed in a dark environment. The samples were placed in an oven set at 60° C. for approximately 2.0 minutes. Tweezers were used to separate the epidermis from the dermis. The separated samples were placed in respective pre-weighed labelled vials and weight of tissues were recorded.

Epidermal and dermal samples were processed and homogenized while maintaining a dark environment using the following protocol: 500 μL of homogenizing mixture, Water:Acetonitrile, 1:1 (v/v), was added to the labelled tubes containing the skin samples. The dermis is homogenized at 10,000 RPM, 4×30 seconds, 45 seconds pause period. The epidermis was then homogenized at 10,000 RPM, 2×30 seconds, 45 seconds pause period. After homogenization is completed, 1000 μL of acetonitrile is added, vortexed briefly, then sonicated for 10 minutes in water bath. The vials were centrifuged and supernatant was used for mass spectrometry analysis. The following samples were analyzed for drug content: Receptor solution samples at different time-points; skin samples (epidermis and dermis for each cell at the final time point), tape strips and cotton swabs analyzed for mass balance determination. The permeation profile of each drug formulation into the receiving fluid and permeation of each drug formulation into the epidermis and dermis at the final time point were calculated.

### Results

Skin retention tests showed that porfimer sodium permeation was predominantly found in the epidermal layers of the skin 24 hours post application (see FIG. 1 and Table 4). No formulations showed porfimer sodium permeation in the receptor fluid.

TABLE 4
In vitro Human Skin Permeation of Porfimer Sodium

| | Applied | Applied | Epidermal | | |
|---|---|---|---|---|---|
| | dose | API Amt | Avg | | % API in |
| Formulation | (mg) | (μg) | (ng/mg) | SD | epidermis |
| 2 | 7.8 | 15.6 | 6.00 | 2.21 | 0.0218 |
| 3 | 7.8 | 15.6 | 11.85 | 5.77 | 0.0481 |
| 4 | 7.8 | 15.6 | 5.20 | 1.68 | 0.0183 |
| 5 | 7.8 | 15.6 | 5.31 | 1.68 | 0.0218 |

Out of the formulations tested, Formulation 3 shows higher epidermal retention at 0.0481% followed by Formulation 2, Formulation 5 and Formulation 4, at 0.0218%, 0.0218% and 0.0183% respectively.

### Example 4: Topical Porfimer Sodium Formulations 7A-7J with or without KI

Sodium porfimer formulations 7A-7J with or without KI were prepared according to Table 5 below.

TABLE 5
Topical Porfimer Sodium Formulations with varying amounts of porfimer sodium with or without KI.

| | | 7A | 7B | 7C | 7D | 7E | 7F | 7G | 7H | 7I | 7J |
|---|---|---|---|---|---|---|---|---|---|---|---|
| Components | Function | % w/w | | | | | | | | | |
| porfimer | API | 0.1 | 0.2 | 0.5 | 0.1 | 0.2 | 0.5 | 0.5 | 0.5 | 0.5 | 0.5 |
| sodium | | | | | | | | | | | |
| Potassium | Potentiator | | | | 1.66* | 1.66* | 1.66* | | 1.66* | | 1.66* |
| Iodide | | | | | | | | | | | |
| Propylene | Solvent | 45-49 | 45-49 | 45-49 | 45-49 | 45-49 | 45-49 | 25-30 | 23-27 | 5-10 | 3-8 |
| glycol | | | | | | | | | | | |
| Permeation | Permeation | 30 | 30 | 30 | 30 | 30 | 30 | 50 total | 50 total | 70 total | 70 total |
| (penetration) | (penetration) | | | | | | | 20 DMSO | 20 DMSO | 40 DMSO | 40 DMSO |
| Enhancer | Enhancement | | | | | | | 30 additional | 30 additional | 30 additional | 30 additional |
| | | | | | | | | penetration | penetration | penetration | penetration |
| | | | | | | | | enhancer | enhancer | enhancer | enhancer |
| Polysorbate 80 | Surfactant | 2.0 | 2.0 | 2.0 | 2.0 | 2.0 | 2.0 | 2.0 | 2.0 | 2.0 | 2.0 |
| Glycerin | Humectant | 15 | 15 | 15 | 15 | 15 | 15 | 15 | 15 | 15 | 15 |
| Benzyl alcohol | Preservative | 2.0 | 2.0 | 2.0 | 2.0 | 2.0 | 2.0 | 2 | 2 | 2 | 2 |
| Phenoxyethanol | Antioxidant | 1.0 | 1.0 | 1.0 | 1.0 | 1.0 | 1.0 | 1 | 1 | 1 | 1 |
| Hexylene glycol | Solvent | 1.0 | 1.0 | 1.0 | 1.0 | 1.0 | 1.0 | 1 | 1 | 1 | 1 |
| Hydroxy propyl | Gelling agent | 1.0 | 1.0 | 1.0 | 1.0 | 1.0 | 1.0 | 1 | 1 | 1 | 1 |
| Cellulose | | | | | | | | | | | |
| Additional | Inactive | Add to | Add to | Add to | Add to | Add to | Add to | Add to | Add to | Add to | Add to |
| Excipients | agent or other | obtain | obtain | obtain | obtain | obtain | obtain | obtain | obtain | obtain | obtain |
| | Filling agent | total w | total w | total w | total w | total w | total w | total w | total w | total w | total w |
| | | of 100 | of 100 | of 100 | of 100 | of 100 | of 100 | of 100 | of 100 | of 100 | of 100 |
| Total | | 100 | 100 | 100 | 100 | 100 | 100 | 100 | 100 | 100 | 100 |

*100 mM KI => 1.66% w/w

### Example 5: Stability Studies on Porfimer Sodium Formulations 7A-7F

Stability studies for porfimer sodium formulations 7A, 7B, and 7C (0.1, 0.2, 0.5% w/w) without KI and porfimer sodium formulations 7D, 7E, and 7F (0.1, 0.2, 0.5 w/w) with KI (100 mM) were conducted.

Environmental chambers were maintained at the following condition temperature (° C.) and relative humidity (RH): Long term storage: 25±2° C./60±5% RH. Refrigerated: 5±3° C. Accelerated storage: 40±2° C./75±5% RH

Each formulation prototype was tested for physical and chemical stability. Physical testing typically included appearance (visual description and color, of the product), pH and microscopy. Chemical testing included, assay (% Label Claim) and impurities

### Results:

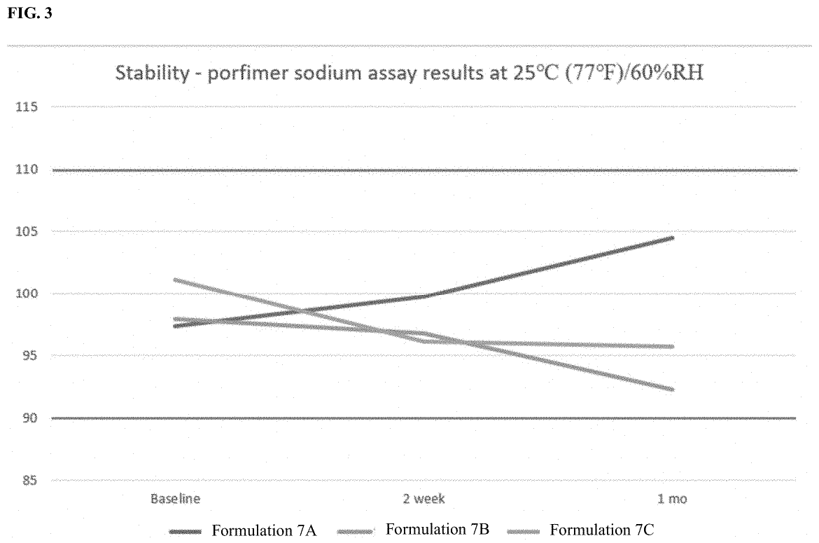

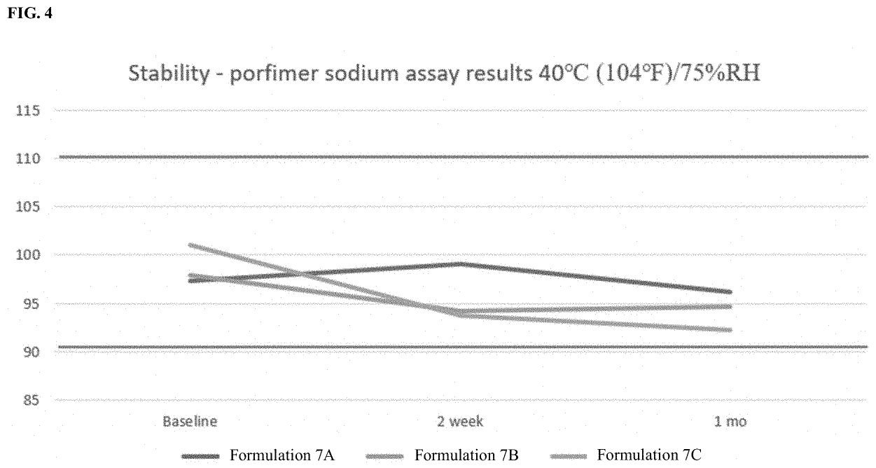

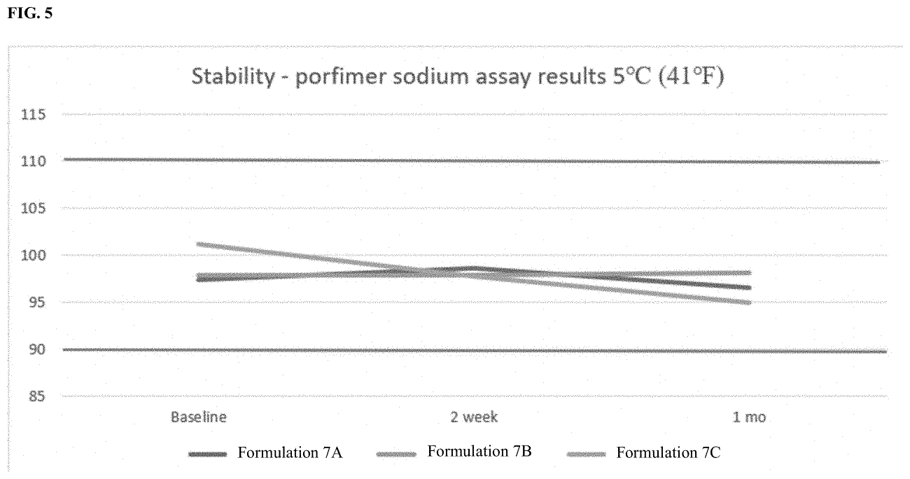

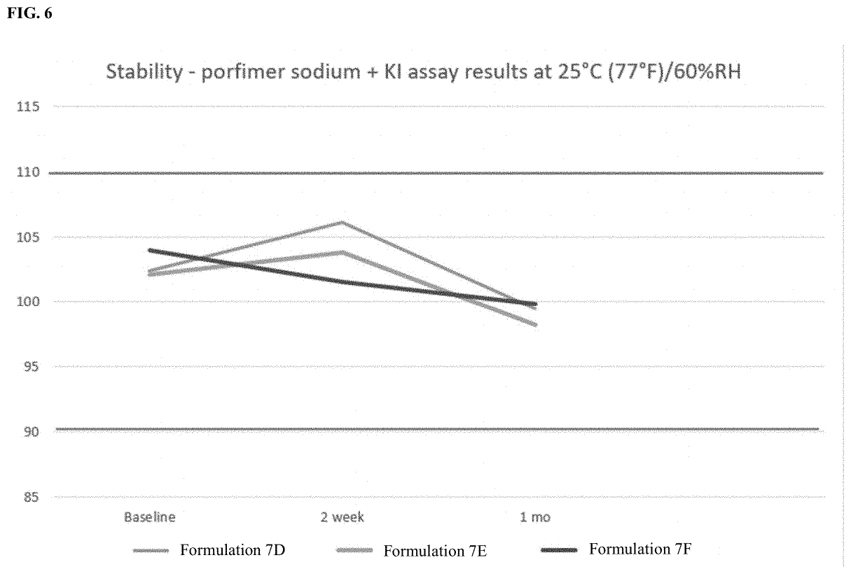

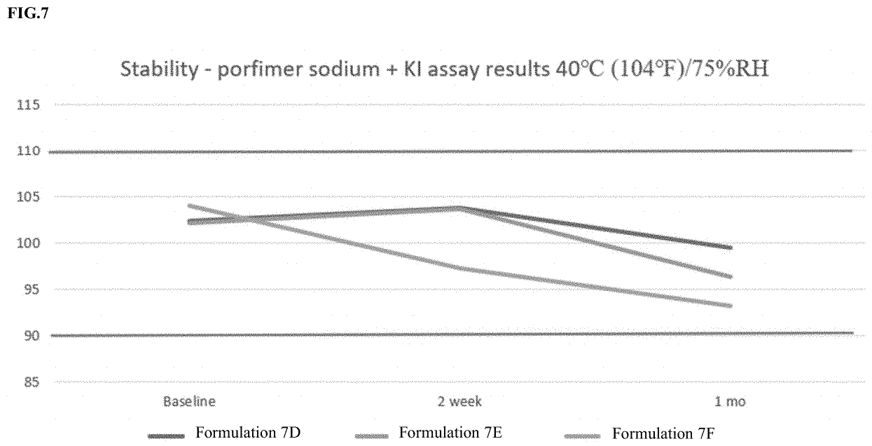

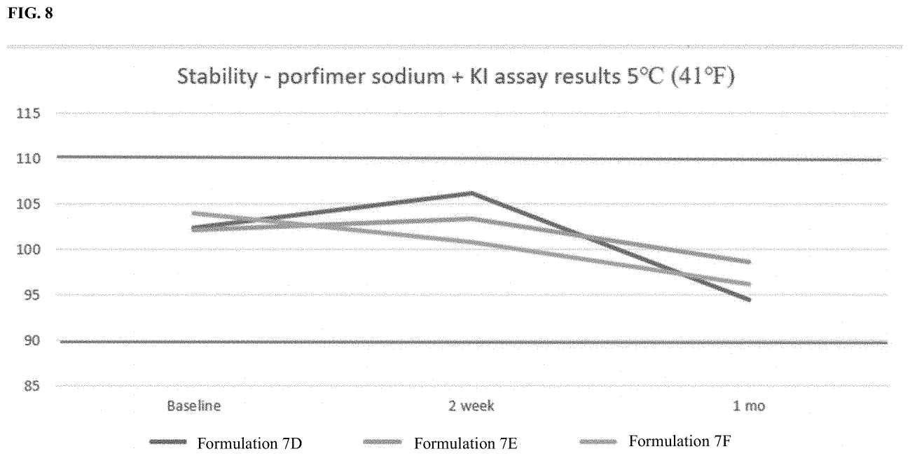

The Stability test results for porfimer sodium formulations 7A-7F stored at 25° C./60% RH, 40° C./75% RH and 5° C. at 2 weeks and 1 month are depicted in FIGS. 3-8. As shown in FIGS. 3-8 the assay values for all samples at two and four weeks was within approximately 93-105% label claim of sodium porfimer.

### Example 6: Study to Assess the Potential Duration and Intensity of Dermal Reaction in Subjects Receiving Topical Administration of a Formulation for the Delivery of Porfimer Sodium with or without Potassium Iodide

This study was designed to assess skin irritation in healthy volunteers following a single topical application of a gel formulation containing porfimer sodium with or without potassium iodide, in variation.

### Study Groups:

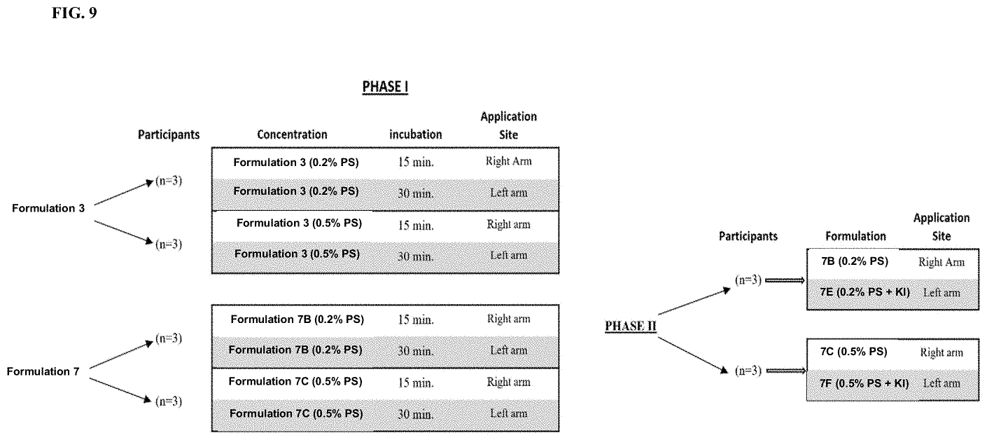

As shown in FIG. 9, in the first set of subjects, two of the variations used were the application of formulation 3 with 2 concentrations of the drug porfimer sodium (0.2% and 0.5%). In the second set of subjects, two of the variations used were a second gel formulation with 2 different concentrations of the drug porfimer sodium 0.2% (formulation 7B) and 0.5% (formulation 7C). In the third set of subjects, two of the variations employed were formulations with porfimer sodium 0.2%+100 mM Potassium Iodide (formulation 7E) and porfimer sodium (0.5%)+100 mM Potassium Iodide (formulation 7F). The study assessed skin irritation after the application of the formulations, an incubation period of either 15 or 30 minutes, followed by light irradiation of 630 nm of approximately 50 J/cm².

### Procedures/Methods:

The photosensitivity procedure was conducted, generally, on the inside of each forearm. The adhesive backed exposure cuff was placed on the skin avoiding, to the extent possible, any cuts, bruises, lesions, freckles, moles or scars. After placing a disposable dose patch a marker was used to identify the coordinates, for future follow-up cuff placement. The formulation was applied to an opening on the cuff and incubated for 30 minutes. Each of the areas was exposed to a light dose (50 J/cm²; approximately 5-minutes). Any sensation that was felt and verbalized by the test subject, was recorded. After the exposure was complete, the areas which had received the topical formulation were wiped with a saline soaked gauze to clean off any remaining or excess formulation. Photo documentation and recording of any visible reaction to the treated area was completed. The data collected consisted of the subject's skin coloration based on an erythema scale and photographic documentation. A visual Analog Scale was be used to collect any information regarding pain associated with the study. Patient's skin response and level and duration of symptoms, if any, were recorded immediately following procedure, 48 hours post, 1-week post and 2 weeks post procedure.

### Results:

There was no report of pain, edema, or pruritis (itch) across all 72 lesions.

Erythema was reported. Pre-treatment, no erythematous lesions were noted for any of the 72 treatment sites in the entire sample. There were also no reports of erythema at 48h, 1 week, or 2 weeks post treatment. All observed erythema was immediate post-exposure only (i.e., transient) (see Tables 6-9) and not accompanied with discomfort (itch or pain). As shown in Table 6, in all groups no ratings above 2 (pink) were noted and, when reported, the most common report was slightly pink (61%).

TABLE 6
Post-treatment Erythema: Immediate all groups

| Erythema | | | Valid | Cumulative |
|---|---|---|---|---|
| Scale | Frequency | Percent | Percent | Percent |
| none | 20 | 27.8 | 27.8 | 27.8 |
| 1 | 44 | 61.1 | 61.1 | 88.9 |
| 2 | 8 | 11.1 | 11.1 | 100.0 |
| Total | 72 | 100.0 | 100.0 | |

Erythema Scale:
0 = no lesion,
1 = slightly pink,
2 = pink,
3 = red and
4 = dark red or purple

TABLE 7
Number of exposed sites in Group 1 that developed erythema post treatment:

| # of | | | Erythema | Erythema | Erythema | Erythema | |
|---|---|---|---|---|---|---|---|
| Sites | | | Scale: 0 | Scale: 0 | Scale: 1 | Scale: 2 | 48 h post |
| Treated | Formulation | Incubation | pre tx | post tx | post tx | post tx | erythema |
| 6 | 3 0.2% PS | 15 m | 6 | 5 | 1 | 0 | 0 |
| 6 | 3 0.2% PS | 30 m | 6 | 4 | 2 | 0 | 0 |
| 6 | 3 0.5% PS | 15 m | 6 | 0 | 5 | 1 | 0 |
| 6 | 3 0.5% PS | 30 m | 6 | 4 | 2 | 0 | 0 |

Erythema Scale:
0 = no color;
1 = slightly pink;
2 = pink;
3 = red;
4 = dark red or purple

TABLE 8
Number of exposed sites in Group 2 that developed ery thema post treatment:

| # of | | | Erythema | Erythema | Erythema | Erythema | |
|---|---|---|---|---|---|---|---|
| Sites | | | Scale: 0 | Scale: 0 | Scale: 1 | Scale: 2 | 48 h post |
| Treated | Formulation | Incubation | pre-tx | post tx | post tx | post tx | erythema |
| 6 | 7A 0.2% PS | 15 m | 6 | 1 | 5 | 0 | 0 |
| 6 | 7A 0.2% | 30 m | 6 | 3 | 3 | 0 | 0 |
| 6 | 7C 0.5% | 15 m | 6 | 1 | 5 | 0 | 0 |
| 6 | 7C 0.5% | 30 m | 6 | 0 | 4 | 2 | 0 |

Erythema Scale:
0 = no color;
1 = slightly pink;
2 = pink;
3 = red;
4 = dark red or purple

TABLE 9
Number of exposed sites in Groups 3 & 4 that developed ery thema post treatment:

| # of | | | Erythema | Erythema | Erythema | Erythema | |
|---|---|---|---|---|---|---|---|
| Sites | | | Scale: 0 | Scale: 0 | Scale: 1 | Scale: 2 | 48 h post |
| Treated | Formulation | Incubation | pre-tx | post-tx | post-tx | post-tx | erythema |
| 6 | 7B 0.2% | 15 m | 6 | 0 | 5 | 1 | 0 |
| 6 | 7E 0.2% + KI | 30 m | 6 | 0 | 6 | 0 | 0 |
| 6 | 7C 0.5% | 15 m | 6 | 0 | 3 | 3 | 0 |
| 6 | 7F 0.5% + KI | 30 m | 6 | 1 | 4 | 1 | 0 |

Erythema Scale:
0 = no color;
1 = slightly pink;
2 = pink;
3 = red;
4 = dark red or purple

### Example 7: Study to Assess the Potential Duration and Intensity of Dermal Reaction in Subjects Receiving Topical Administration of a Formulation for the Delivery of Porfimer Sodium with or without Potassium Iodide

This study was designed to assess skin irritation in healthy volunteers following a single topical application of a gel formulation containing porfimer sodium with or without potassium iodide, in variation.

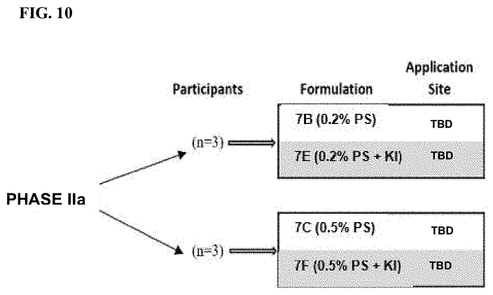

The study assessed skin irritation after the application of the formulations, an incubation period of 30 minutes, followed by light irradiation of 630 nm of approximately 50 J/cm². In this sample, 6 subjects were given the formulations as described in Example 6 (formulations 7B, 7C, 7E and 7F), however test sites were on various locations of the body of the subject where there was evidence of chronic sun exposure, including evidence of seborrheic keratosis and actinic keratosis. See FIG. 10.

### Procedures/Methods:

The photosensitivity procedure was conducted on various location of the body where there was evidence of chronic sun exposure, such as wrinkling, pigment spots and/or seborrheic keratosis and actinic keratosis. The adhesive backed exposure cuff was placed on the skin avoiding, to the extent possible, any cuts, bruises, lesions, freckles, moles or scars. After placing a disposable dose patch a marker was used to identify the coordinates, for future follow-up cuff placement. The formulation was applied to an opening on the cuff and incubated for 30 minutes. Each of the areas was exposed to a light dose (50 J/cm²; approximately 5 minutes). Any sensation that was felt and verbalized by the test subject, was recorded. After the exposure was complete, the areas which had received the topical formulation were wiped with a saline soaked gauze to clean off any remaining or excess formulation. Photo documentation and recording of any visible reaction to the treated area was completed. The data collected consisted of the subject's skin coloration based on an erythema scale and photographic documentation. A visual Analog Scale was be used to collect any information regarding pain associated with the study. Patients skin response and level and duration of symptoms, if any, were recorded immediately following procedure, 48 hours post, 1-week post and 2 weeks post procedure.

Specifically, 24 areas on 6 subjects were included in this study. Of those 19 areas were treated with a formulation and 630 nm light as follows: 4 areas received 0.2% PS (formulation 7B); 6 areas received 0.2% PS+KI (formulation 7E); 4 areas received 0.5% PS (formulation 7C); and 5 areas received 0.5% PS+KI (formulation 7F). A total of 5 areas received light (630 nm) only as a control. Of the non-control areas for administration, six areas had seborrheic keratosis, ten areas had actinic keratosis, two areas had evidence of sun exposed skin, one area had a small pigmented spot (lentigo).

TABLE 10
Post-Treatment Side Effects of Pruritis, Pain or Edema

| | # of sites | Evidence of | Evidence of | Evidence |
|---|---|---|---|---|
| Formulation | treated | Pruritis | Pain | of Edema |
| Formulation | 4 | None reported | Reports of mild | None |
| 7B | | | pain at the | |
| | | | immediate post | |
| | | | time point in | |
| | | | 2 of 4 areas | |
| | | | (same subject) | |
| Formulation | 6 | Reports of | Reports of mild | 1 of 6 areas |
| 7E | | pruritis | pain at the | demonstrated |
| | | immediately | immediate post | edema |
| | | post light | time point in | immediately |
| | | exposure in | 1 of 6 areas | post light |
| | | 2 of 6 | (same subject | exposure |
| | | areas | reporting pain | |
| | | | with formula- | |
| | | | tion 7B) | |
| formulation | 4 | None reported | None reported | None |
| 7C | | | | |
| formulation | 5 | None reported | None reported | None |
| 7F | | | | |

As shown above, the subjects had minimal to no adverse side effects of pruritis, pain or edema. Of the 6 subjects, only one subject perceived very mild pain in three different treatment areas. One subject had transient edema in one treated site of actinic keratosis.

That post administration increased the erythema on the wound or lesion area before treatment.

TABLE 11
Post-Treatment Side Effects of Erythema

| | | Areas | Increased | Increased | Increased | Increased |
|---|---|---|---|---|---|---|
| | | with | Erythema | Erythema | Erythema | Erythema |
| | # of | Erythema | Immediate | 48 Hrs | 1 Week | 2 Weeks |
| | sites | Pre | Post | Post | Post | Post |
| Formulation | treated | Treatment | Treatment | Treatment | Treatment | Treatment |
| Formulation 7B | 4 | 1/4 | 1/4 | 0/4 | 0/4 | 0/4 |
| Formulation 7E | 6 | 2/4 | 4/6 | 3/6 | 0/6 | 0/6 |
| formulation 7C | 4 | 1/4 | 3/4 | 3/4 | 0/4 | 0/4 |
| formulation 7F | 5 | 4/5 | 2/5 | 2/5 | 0/5 | 0/5 |
| Control | 5 | 0 | 1/5 | 0 | 0 | 0 |
| (light only) | | | | | | |

As shown above, all increased lesional and non lesional erythema subsided post 48 hours.

### Results:

All subjects completed the study. It is important to note particularly in the assessment of the areas treated in the patients that the erythema that was manifested was mainly confined to the lesion within the treated area (with the exception of one lesion in a single subject). This was the case for those lesions described as actinic keratosis lesions and seborrheic keratosis lesions. This demonstrates that with what would be considered to be sub-threshold light and drug dose combinations, there was a somewhat selective effect on the lesion within a treated area with little adverse side effects outside of the lesion or treated area.

Furthermore, with the exception of one subject (1 of 6 subjects), there were no reports of treatment associated pain in this study. When added to the first 18 patients from example 6, this supports the hypothesis that the formulations at the two porfimer sodium concentrations tested (0.2% and 0.5%) with and without the addition of potassium iodide (KI) did not result in pain at the treated site. In the subject that did report pain in 3 of 4 sites treated, in the chronically sun exposed group, the VAS score was 1 in (2) lesions and 2 in (1) lesion during therapy. Immediately post therapy the subject reported pain in 2 lesions with a score of 1 and the pain in the third lesion had resolved immediately after the light was removed. Accordingly, in this case, the pain sensation was tolerable and did not interfere with the conduct of the light exposure.

### Conclusions:

Very few if any subjects had any sensitivity from the study parameters used. One subject perceived very mild pain and one subject had transient edema in one treated site of actinic keratosis.

Also, all increased lesional and non lesional erythema subsided just over 48 hours. Furthermore, there was evidence of rapid treatment, as one pigmented lesion resolved by 50% and one actinic keratosis completely resolved at three-week follow up visit. Examples 6 and 7 thus show the ability to treat chronically exposed skin, without or mild adverse side effects such as pain and increased erythema.

### Example 8: Study to Assess the Treatment of Actinic Keratosis in Subjects Receiving Topical Administration of a Formulation for the Delivery of Porfimer Sodium

The application of Formulations 7B and 7C with 2 concentrations of the drug porfimer sodium (0.2% and 0.5%) is applied to a set of subjects with actinic keratosis. The study assesses the intensity of dermal reaction of actinic keratosis after the application of the formulations with an incubation period of 30 minutes, followed by light irradiation of 630 nm of approximately 50 J/cm² over 5 minutes.

### Procedures/Methods:

The procedure is conducted, generally to expose actinic keratosis on the subject to photodynamic therapy with formulations 7B or 7C with topical porfimer sodium. Photodynamic therapy is carried out to treat lesions on the upper extremities including the arms, chest or head. The formulations are applied to the selected area and incubated for 30 minutes. Each of the areas is exposed to a light dose (50 J/cm²; approximately 5 minutes). Any sensation that is felt and verbalized by the test subject, is recorded. After the exposure is complete, the areas which received the topical formulation are wiped with a saline soaked gauze to clean off any remaining or excess formulation and the erythema scoring recorded. Photo documentation and recording of any visible reaction and improvement of symptoms to the treated area is completed after treatment.

### Results:

Resolution of the actinic keratoses or reduction of the appearance of actinic keratosis is achieved by the subjects receiving the formulation 7B and 7C with porfimer drug. Furthermore, there is no difference in pain, edema, erythema and/or pruritis between subjects receiving formulation with porfimer drug and not receiving porfimer drug. The present disclosure thus provides a method for treating actinic keratoses without the adverse side effects commonly known in the art.

### Example 9: Study to Assess the Treatment of Chronically Sun Exposed Skin and Seborrheic Keratosis Receiving Topical Administration of a Formulation for the Delivery of Porfimer Sodium without Potassium Iodide

The application of Formulations 7B and 7C in 2 concentrations of the drug porfimer sodium (0.2% and 0.5%) is applied to a set of subjects with photo-damaged skin, such as chronically sun exposed skin and seborrheic keratosis. The study assesses the treatment of photo-damaged skin after the application of the formulations with an incubation period of 30 minutes, followed by laser light irradiation of 630 nm of approximately 50 J/cm² for 5 minutes.

### Procedures/Methods:

The procedure is conducted, generally to treat photo-damaged skin on the subject. Photodynamic therapy with formulation 7B or 7C with porfimer sodium is carried out to treat photo-damaged skin on the upper extremities in patients with mild to moderate photo-damaged skin from chronic sun exposure, such as wrinkles or pigment spots, or seborrheic keratosis. Formulations are applied and incubated for 30 minutes. Each of the areas is exposed to a light dose (50 J/cm²; approximately 5 minutes 630 nm). Any sensation that was felt and verbalized by the test subject, is recorded. After the exposure is complete, the areas which received the topical formulation are wiped with a saline soaked gauze to clean off any remaining or excess formulation. Photo documentation and recording of any visible reaction and improvement of symptoms to the treated area is completed after treatment.

### Results:

Areas treated with pigmented lesions within the chronically sun exposed skin demonstrate an at least 50% lightening of the lesion (seborrheic keratosis) upon follow-up. The lesions also generally demonstrate an increase in erythema score of 1 which resolved within 48 hours post exposure. Reduction of photo-damaged skin, such as pigment spots or wrinkles is also achieved in the subjects receiving the formulation with porfimer drug. Furthermore, there is no difference in pain, edema, erythema and/or pruritis between subjects receiving formulation and no formulation. The present disclosure thus provides a method for treating photo-damaged skin and seborrheic keratosis without the adverse side effects commonly known in the art.

### Example 10: Study to Assess the Treatment of Actinic Keratosis in Subjects Receiving Topical Administration of a Formulation for the Delivery of Porfimer Sodium with Potassium Iodide

The application of formulations with porfimer sodium 0.2%+100 mM Potassium Iodide (formulation 7E) and porfimer sodium (0.5%)+100 mM Potassium Iodide (formulation 7F), is applied to a set of subjects with actinic keratosis. The study assesses the exposure of actinic keratosis after the application of the formulations with an incubation period of 30 minutes, followed by light irradiation of 630 nm of approximately 50 J/cm² over 5 minutes.

### Procedures/Methods:

The procedure is conducted, generally to treat actinic keratosis on the subject. Photodynamic therapy with a Formulation 7 E or F, is carried out to treat lesions on the upper extremities with actinic keratoses. The formulation is applied and incubated for 30 minutes. Each of the areas is exposed to a light dose (50 J/cm²; approximately 5-12 minutes). Any sensation that is felt and verbalized by the test subject, is recorded. After the exposure is complete, the areas which receive the topical formulation is wiped with a saline soaked gauze to clean off any remaining or excess formulation. Photo documentation and recording of any erythema or improvement of symptoms on subsequent follow-up to the treated area is completed after each treatment session.

### Results:

Resolution of the actinic keratoses or reduction of the appearance of actinic keratosis is achieved by the subjects receiving the formulation 7E and 7F with porfimer drug. Furthermore, there is no difference in pain, edema, erythema and/or pruritis between subjects receiving formulation with porfimer drug and without. The present disclosure thus provides a method for treating actinic keratoses without the adverse side effects commonly known in the art.

### Example 11: Study to Assess the Treatment of Chronically Sun Exposed Skin and Seborrheic Keratosis Receiving Topical Administration of a Formulation for the Delivery of Porfimer Sodium with Potassium Iodide

The application of formulations with porfimer sodium 0.2%+100 mM Potassium Iodide (formulation 7E) and porfimer sodium (0.5%)+100 mM Potassium Iodide (formulation 7F) is applied to a set of subjects with photo-damaged skin. The study assesses the treatment of photo-damaged skin after the application of the formulations with an incubation period of 30 minutes, followed by light irradiation of 630 nm of approximately 50 J/cm² over 5 minutes.

### Procedures/Methods

The procedure is conducted, generally to treat photo-damaged skin on the subject. Photodynamic therapy with a formulation 7E or 7F with porfimer and potassium iodide is carried out to treat photo-damaged skin on the upper extremities with mild to moderate photo-damaged skin, such as wrinkles or pigment spots, or seborrheic keratosis. The formulation is applied and incubated for 30 minutes. Each of the areas is exposed to a light dose (50 J/cm²; approximately 5 minutes). Any sensation that is felt and verbalized by the test subject, is recorded. After the exposure is complete, the areas which received the topical formulation is wiped with a saline soaked gauze to clean off any remaining or excess formulation. Photo documentation and recording of any visible reaction and improvement of symptoms to the treated area is completed after each treatment.

### Results:

Areas treated with pigmented lesions within the chronically sun exposed skin demonstrate an at least 50% lightening of the lesion (seborrheic keratosis) upon follow-up. The lesions also generally demonstrate an increase in erythema score of 1 which resolved within 48 hours post exposure. Reduction of photo-damaged skin, such as pigment spots or wrinkles is also achieved in the subjects receiving the formulation with porfimer drug. Furthermore, there is no difference in pain, edema, erythema and/or pruritis between subjects receiving formulation and no formulation.

The present disclosure thus provides a method for treating photo-damaged skin and seborrheic keratosis without the adverse side effects commonly known in the art.

### Example 12: Study to Assess the Potential Duration and Intensity of Dermal Reaction in Subjects Receiving Topical Administration of a Formulation for the Delivery of Porfimer Sodium with DMSO and with or without Potassium Iodide

This study is designed to assess skin irritation in healthy volunteers following a single topical application of a gel formulation containing porfimer sodium with DMSO and with or without potassium iodide, in variation.

### Study Groups

The potential duration and intensity of dermal reaction of porfimer sodium formulations (Formulations 71 and 7J of Example 4, Table 5) are tested. Subjects in the test groups receive: (i) a formulation with porfimer sodium 0.5% wt+dimethyl sulfoxide (DMSO) 40% wt+diethylene glycol monoethyl ether (DEGEE) 30% wt (formulation 71); or (ii) a formulation with porfimer sodium 0.5% wt+DMSO 40% wt+DEGEE 30% wt+100 mM potassium iodide 1.66% wt (formulation 7J). The formulation is applied on various locations of the body of the subject where there is evidence of chronic sun exposure. Subjects in the placebo group receive a gel formulation without porfimer sodium. The study assesses skin irritation following the application of the formulations and an incubation period of either 15 or 30 or 60 minutes followed by light irradiation of 630 nm with approximately 50 J/cm² or 100 J/cm².

### Procedures/Methods

The photosensitivity procedure is conducted, generally, on the inside of each forearm. The adhesive backed exposure cuff is placed on the skin avoiding, to the extent possible, any cuts, bruises, lesions, freckles, moles or scars. After placing a disposable dose patch a marker is used to identify the coordinates, for future follow-up cuff placement. The formulation is applied to an opening on the cuff and incubated for 30 minutes. Each of the areas is exposed to a light dose (50 J/cm² or 100 J/cm²; approximately 6-12 minutes). Any sensation that is felt and verbalized by the test subject, is recorded. After the exposure is complete, the areas which had received the topical formulation are wiped with a saline soaked gauze to clean off any remaining or excess formulation. Photo documentation and recording of any visible reaction to the treated area is completed. The data collected consisted of the subject's skin coloration based on an erythema scale and photographic documentation. A visual Analog Scale is used to collect any information regarding pain associated with the study. Patients skin response and level, duration of symptoms, and safety data were recorded immediately following procedure, 48 hours post, 1-week post and 2 weeks post procedure.

### Inclusion Criteria

Subjects must satisfy all of the following criteria:

Female and Male healthy volunteers 18-75 years of age; Can understand and communicate effectively in English and can provide written consent; Have Fitzpatrick skin type I-IV; Have healthy and chronically sun exposed skin (with or without evidence of actinic keratosis) on the area(s) to be tested with a minimal area or number of sites to accommodate each formulation and light dose; and Capable of giving written informed consent, which includes compliance with the requirements and restrictions listed in the consent form.

### Exclusion Criteria

Subjects are excluded from the study for any of the following reasons: Current use of commercial tanning equipment; Have Fitzpatrick skin types V-VI; Have skin wounds or lesions at the site of the planned exposure; Have current skin cancer or personal history of skin cancer; Have diabetes, psoriasis, or other inflammatory skin condition; Current use of steroids, corticosteroids, inhaled steroids for asthma; Less than 18 years of age or older than 75 years of age; Unable to provide informed consent; Have history of porphyria or other endogenous photosensitivity disease; Presence of tattoos on the inner forearms.

### Example 13: In Vivo Evaluation of the Ability of Porfimer Sodium Gel Formulations with or without DMSO and with or without 100 mM KI to Reduce/Eliminate Microbial Colonization in a Partial Thickness Wound Healing Model

This study evaluates the ability of three different concentrations of porfimer sodium (0.1%, 0.2% and 0.5%) in a gel formulation with (Formulations 7D-F, 7H) or without 100 mM KI (Formulations 7A-C, 7G) to reduce/eliminate microbial colonization from surgical sites in a partial thickness wound healing model. This study also evaluates the ability of porfimer sodium in a gel formulation with 20% concentration of DMSO (Formulations 7G-H with 20% wt DMSO).

The study is conducted in female Yorkshire pigs over the course of 4 days. A Dermatome is used to create partial thickness wounds of approximately 2.0 cm×2.0 cm in width and length with a depth of 0.5 mm parallel to the spine of the animal with one row of five (5) wounds along each side of the animal for the test article treatment group animals and or one row of eight (8) and a second row of seven (7) wounds along the opposite sides of the control group animal.

Designated wounds on each of the animals are infected with methicillin-resistant Staphylococcus aureus (MRSA-ATCC 1768) following wound creation. Three (3) wounds on the control group animal are designated as uninfected controls and are not inoculated. The wounds are bandaged, and bacterial colonization is allowed to develop overnight. Designated infected wounds in the test article treatment groups are subjected to a single course of treatment. Treatments consist of application of the test article followed by an incubation period of 30 minutes then photoactivation at 200 J/cm² for 20 minutes. Designated infected wounds in the control group receive application of the test article but are not subjected to photoactivation. Microbial load is determined from wound swabs taken just prior to treatment, one hour and 24 hours following treatment, and tissue biopsies are harvested at 24 hours after treatment. Experiments are conducted under dim light (less than 200 lux). The treatment Groups are summarized in Table 13 below.

TABLE 12
Treatment Groups

| | | | Bacteria | Vol. | |
|---|---|---|---|---|---|
| | | | (10⁶ | (cc's) | Light |
| | | Number of | to 10⁷ | formula- | intensity |
| Treatment | Group | lesions | CFU/wound) | tion/group | (J/cm²) |
| Uninfected/ | 1 | 3 | N/A | 0 | N/A |
| Untreated | | | | | |
| Infected/ | 2 | 3 | S. aureus | 0 | N/A |
| Untreated | | | ATCC BAA | | |
| | | | 1768 | | |
| Infected/ | 2a | 3 | S. aureus | 3 | N/A |
| Placebo no | | | ATCC BAA | | |
| PS + KI | | | 1768 | | |
| Infected/ | 2b | 3 | S. aureus | 3 | N/A |
| Placebo no | | | ATCC BAA | | |
| PS no KI | | | 1768 | | |
| Infected/ | 2c | 3 | S. aureus | 3 | N/A |
| Placebo + | | | ATCC BAA | | |
| PS* no KI | | | 1768 | | |
| Infected/ | 3 | 5 | S. aureus | 5 | 200 |
| Formulation | | | ATCC BAA | | |
| 7C | | | 1768 | | |
| Infected/ | 3 | 5 | S. aureus | 5 | 200 |
| Formulation | | | ATCC BAA | | |
| 7F | | | 1768 | | |
| Infected/ | 3 | 5 | S. aureus | 5 | 200 |
| Formulation | | | ATCC BAA | | |
| 7G | | | 1768 | | |
| Infected/ | 3 | 5 | S. aureus | 5 | 200 |
| Formulation | | | ATCC BAA | | |
| 7H | | | 1768 | | |
| Infected/ | 4 | 5 | S. aureus | 5 | 200 |
| Formulation | | | ATCC BAA | | |
| 7A | | | 1768 | | |
| Infected/ | 4 | 5 | S. aureus | 5 | 200 |
| Formulation | | | ATCC BAA | | |
| 7D | | | 1768 | | |
| Infected/ | 5 | 5 | S. aureus | 5 | 200 |
| Formulation | | | ATCC BAA | | |
| 7B | | | 1768 | | |
| Infected/ | 5 | 5 | S. aureus | 5 | 200 |
| Formulation | | | ATCC BAA | | |
| 7E | | | 1768 | | |

*The concentration of PS (porfimer sodium) in the placebo groups is 0.2%

### Example 14: Study to Assess the Porfimer Sodium with or without Potassium Iodide with or without DMSO to Reduce/Eliminate Microbial Colonization and Biofilms in Partial Thickness Wounds

Sodium porfimer formulations 71 and 7J with or without KI are prepared according to Table 5 in Example 4.

Partial thickness wounds are created parallel to the spine of pig subjects for a total of 28 partial thickness wounds all of are infected with methicillin resistant S. aureus ATCC BAA 1768 and the untreated or treated in a different treatment group as provided below in Table 13.

TABLE 13
Treatment Groups for Partial Thickness
Wounds in Pig Study Groups

| | | | | Bacteria | |
|---|---|---|---|---|---|
| | | | | (10⁶ | Light |
| | | | Number of | to 10⁷ | intensity |
| Treatment | Group | Animal | lesions | CFU/wound) | (J/cm²) |
| Infected/ | 1 | 1 | 2 | S. aureus | N/A |
| Untreated | | | | ATCC BAA | |
| | | | | 1768 | |
| Infected/ | 2 | 1 | 3 | S. aureus | N/A |
| Placebo | | | | ATCC BAA | |
| | | | | 1768 | |
| Infected/ | 3 | 1 | 3 | S. aureus | 200 |
| Formulation | | | | ATCC BAA | |
| 7I | | | | 1768 | |
| Infected/ | 4 | 1 | 3 | S. aureus | 200 |
| Formulation | | | | ATCC BAA | |
| 7J | | | | 1768 | |
| Infected/ | 5 | 1 | 3 | S. aureus | 200 |
| Photofrin | | | | ATCC BAA | |
| HPC 0.5% | | | | 1768 | |

One hundred microliters (100 μL) of freshly cultured methicillin-resistant Staphylococcus aureus (ATCC BAA 1768) at a concentration of 10⁷ to 10⁸ CFU/mL are spotted into the center of each wound in all wounds using a sterile pipet. The bacterial inoculum is lightly scrubbed into the test site for 10 seconds using a sterile spatula (maintaining care to limit the exposure to the wound and not exposing the normal skin adjacent to the wound with the infectious agent) and allow to dry for 3 minutes. The procedures of the study are further provided below in Table 14.

Application of formulations will be to specified legions with subsequent light treatment for 5 days. Swab samples are taken at pre application of formulation, one hour post light treatment and 24 hours post light treatment. The 24-hour swab samples also serve as the pre-treatment swab sample on days 2, 3, 4, and 5. Twenty-four hours post treatment day 5, swab samples are taken followed by biopsy samples of each lesion.

TABLE 14
Schedule of Procedures

| Study Day | | | |
|---|---|---|---|
| (dates) | Event | | Detail |
| Day −1 | Surgery/ | Animal 1 | 14 partial thickness |
| | Administration | | wounds up to 2.0 cm- |
| | of bacteria | | 2.0 cm each, incised on |
| | | | each animal. |
| | | | Bacteria instilled into all |
| | | | wounds on both animals. |
| Day 0 | Treatment | Experiments to | Pre-treatment swab of |
| | | be conducted | all wounds for CFU = |
| | | under dim light | 14 swabs for plating |
| | | (less than 200 | Apply specific test |
| | | lux) | article formulation to |
| | | | designated Treatment |
| | | | Groups. No Test article |
| | | | (formulation) is to be |
| | | | applied to group 1 |
| | | | animal 1. |
| | | | Incubate drug for 30 |
| | | | minutes followed by 16 |
| | | | min and 40 sec exposure |
| | | | with 200 J/cm² light. |
| | | | Obtain swabs of each of |
| | | | the treated wound for |
| | | | bacterial load estimate at |
| | | | 1 h post light |
| | | | treatment. 12 swabs for |
| | | | plating |
| | | | Obtain swabs from |
| | | | group 1 at 1 hour post |
| | | | treatment swab for |
| | | | bacterial load estimate. 2 |
| | | | swabs for plating |
| Day 1 | Course #2 | Formulation | Obtain 24-hour post- |
| | Treatment | application and | treatment swabs for |
| | | experiments to be | all lesions on animal |
| | | conducted under | 1. 14 swabs for |
| | | dim light (less | plating. |
| | | than 200 lux) | Apply photofrin |
| | | | formulation to lesions |
| | | | in groups as |
| | | | designated. Incubate |
| | | | for 30 min. |
| | | | In order, treat all |
| | | | lesions with 200 |
| | | | J/cm² of light. |
| | | | Obtain swabs of each |
| | | | of the treated wound |
| | | | for bacterial load |
| | | | estimate at 1 h post |
| | | | light treatment. 14 |
| | | | swabs for plating. |
| Day 2-4 | Courses | Formulation | Obtain 24-hour post- |
| | 3-5. Repeat | application | treatment swabs for |
| | treatment | and | all lesions. 42 swabs |
| | course | experiments | (3 days). |
| | | to be | Apply photofrin |
| | | conducted | formulation to lesions |
| | | under dim | in groups as |
| | | light (less | designated. Incubate |
| | | than 200 lux) | for 30 min. |
| | | | In order, treat all |
| | | | lesions with 200 |
| | | | J/cm² of light. |
| | | | Obtain swabs of each |
| | | | of the treated wound |
| | | | for bacterial load |
| | | | estimate at 1 h post |
| | | | light treatment. 42 |
| | | | swabs for plating (3 |
| | | | days) |
| Day 6 | Tissue | Procedures to | Euthanize |
| | collection | be conducted | Swab of all wounds for |
| | for biopsy | under dim | CFU. 14 swabs for |
| | and microbial | light (less | plating |
| | load. | than 200 lux) | Harvest wound tissues - |
| | | | ½ for histology. ½ for |
| | | | CFU). 14 tissues for |
| | | | plating |

The respective Photofrin formulation (71 and 7) is applied to the wound bed and incubated for times as indicated in Table N. The formulation is then activated in each wound following application using lens fiber optic attached to a 630-nm emitting laser with 200 J/cm² (approximately between 20 to 25 minutes). The wounds are then swabbed at 1 hour post treatment and will then be rebandaged and wrapped.

24 hours following the treatment as in Table 14, the animals are euthanized and the bandages are removed and the wounds will be swabbed for determination of microbial load. Wound tissues are harvested with one half of the harvested tissue placed into 10% formalin for histology and the other half will be placed into a pre-weighed culture tube containing sterile PBS for determination of microbial load.

Wounds are treated, surgical sites are examined, and bandages are changed on the days of treatment indicated in Table 14. All procedures re performed under ABSL-2 conditions. On each day of the of treatment, the outer bandaging is removed using sterile scissors and the bandages are removed using sterile forceps. Prior to treatment, each wound is swabbed using a sterile cotton tipped applicator in a serpentine motion starting at the upper left hand margin of the wound (relative to the spine) and ending at the bottom right wound margin. The swabs are placed into a sterile culture tube containing 1 to 2 mL of sterile PBS for determination of microbial load.

For determination of microbial load, the tubes containing PBS and wound are vortexed and serial dilutions of the eluted bacteria are prepared in sterile PBS and plated onto Oxacillin Screen Agar (Mueller Hinton Agar with 6 μg/mL Oxacillin and 4% NaCl) in duplicate. Colonies on the plates are enumerated following incubation at 37° C. and the bacterial load is reported as colonies forming units per milliliter of wound fluid (CFU/mL).

The tubes containing the tissue specimens are weighed to determined tissue mass. Wound tissues are homogenized and serial dilutions of the homogenates are prepared in PBS and plated onto Oxacillin Screen Agar in duplicate. Colonies on the plates will be enumerated following incubation at 37° C. and the bacterial load will be reported as colonies forming units per milligram of wound tissue (CFU/mg).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A pharmaceutical composition comprising a photosensitizer and one or more gelling agents.

2. The pharmaceutical composition of claim 1, wherein the photosensitizer is selected from one or more of the group consisting of porphyrins, chlorins (HPPH; NPe6; Temoporfrin (Foscan), mTHPC)), and porphysomes as in: pyropheophorbide nanovesicles including, bacteriochlorophyll porphysomes, zinc pyropheophorbide porphysomes and pyropheophyorbide porphysomes, and chlorin-like compounds (benzoporphyrin; Verteporfin, bacteriochlorins and phthalocyanines, purpurins (tin ethyl etiopurpurin); Metalloporphyrins (Texaphyrins); Pheophorbides (TOOKAD); Protoporphyrins (Levulan, Metvix, 5-ALA (PpIX)) and nonporphyrin based photosensitizers including phenothiazinium salts such as Methylene Blue, Toluidine Blue, Nile Blue, Cyanines, hypericin and Chalcogenpyrilium dyes; PPA904; benzophenothiazinium dye EtNBS; the xanthene class of fluorescent dyes that includes fluorescein and Rose Bengal; Fullerenes (C60 fullerene coupled to polar diserinol groups or quaternary pyrrolidinium groups); Squaraogenines, BODIPY (boron-dipyrromethene) dye, Phenalenones; Hypericin, Hypocrellin, Riboflavin, Curcumin, Titanium dioxide and porfimer sodium (Photofrin®).

3. The pharmaceutical composition of claim 1, wherein the photosensitizer is porfimer sodium.

4. The pharmaceutical composition of claim 3, wherein the porfimer sodium is in an amount ranging from about 0.01% wt to about 1.0% wt; or about 0.05% wt to about 0.7% wt; or about 0.1% wt to about 0.5% wt; or about 0.15% wt to about 0.3% wt.

5. The pharmaceutical composition of claim 3, wherein the porfimer sodium is an amount ranging from 0.4% wt to 0.6% wt.

6. The pharmaceutical composition of any one of claims 1-5, wherein the gelling agent is selected from one or more of the group consisting of a, carbomer, crosslinked, polyacrylic acid, lecithin such as Lecithin-PLO, hydroxyethyl cellulose, hydroxypropyl cellulose, and hydroxypropyl methylcellulose

7. The pharmaceutical composition of claim 6, wherein at least one gelling agent is a carbomer.

8. The pharmaceutical composition of claim 7, wherein the gelling agent is a polymer of acrylic acid cross-linked with polyalkenyl ethers or divinyl glycol.

9. The pharmaceutical composition of claim 7, wherein the carbomer is a Carbopol polymer.

10. The pharmaceutical composition of any one of claims 1-9, wherein the gelling agent is in an amount ranging from about 0.5% wt to about 3.0% wt, or from about 0.7% wt to about 2.0% wt, or from about 0.8% wt to about 1.2% wt.

11. The pharmaceutical composition of any one of claims 1-9, wherein the gelling agent is in an amount ranging from about 1% wt.

12. The pharmaceutical composition of any one of claims 1-10, wherein the pharmaceutical composition further comprises one or more permeation enhancers.

13. The pharmaceutical composition of claim 7, wherein the permeation enhancer is selected from one or more of the group consisting of propylene glycol SR, polyethylene glycol 400 SR, polyethylene glycol 300 LA, diethylene glycol monoethyl ether, dimethyl sulfoxide (DMSO), and Polysorbate 80 SR.

14. The pharmaceutical composition of claim 13, wherein the one or more permeation enhancers selected from the group consisting of propylene glycol SR, dimethyl sulfoxide, and diethylene glycol monoethyl ether.

15. The pharmaceutical composition of any one of claims 12-14 wherein the one or more permeation enhancers are in an amount ranging from about 55.0% wt to about 85% wt, or about 60% wt to about 80% wt, or about 65% wt to about 75% wt.

16. The pharmaceutical composition of any one of claims 12-15, wherein the pharmaceutical composition comprises a photosensitizer, a gelling agent, one or more permeation enhancers, a humectant, a solubilizer/surfactant, and a preservative.

17. The pharmaceutical composition of claim 16, wherein:
i) the humectant is in an amount ranging from about 10% wt to about 20% wt, or about 15% wt;
ii) the solubilizer/surfactant is in an amount ranging from 1.0% wt to about 5% wt, or about 1% to about 3%, or about 2%; and/or
iii) the preservative is in an amount ranging from 0.5% wt to about 5% wt, or about 0.5% wt to about 2% wt, or about 1% wt to about 3% wt.

18. The pharmaceutical composition of any one of claims 1-17, wherein the pharmaceutical composition is a topical formulation.

19. The pharmaceutical composition of claim 18, wherein the topical formulation comprises porfimer sodium and when applied to human skin under the conditions as provided in Example 7, provides at least 0.03% of epidermal retention of the porfimer.

20. The pharmaceutical composition of claim 18, wherein the topical formulation comprises porfimer sodium and when applied to woundless human skin of a subject, provides at least 0.03% percent of the porfimer in the epidermal-dermal layer of the subject.

21. The pharmaceutical composition of claim 19 or 20, wherein the composition, after 40° C./75% RH for 4 weeks, has a total impurities less than 20% by HPLC.

22. A pharmaceutical topical formulation comprising porfimer sodium and one or more pharmaceutical acceptable excipients, wherein when applied to the infected area, or wound of a subject, provides at least 0.03% of the porfimer in the dermis of the subject.

23. The pharmaceutical topical formulation of claim 19, wherein the topical formulation provides at least 0.04% of epidermal retention of the porfimer

24. The pharmaceutical topical formulation of claim 22 or 23, wherein the composition, after 40° C./75% RH for 4 weeks, has a total impurities less than 20% by HPLC.

25. The pharmaceutical topical formulation of any one of claims 22-24, wherein the pharmaceutical topical formulation further comprises a gelling agent, one or more permeation enhancers.

26. A pharmaceutical composition comprising a photosensitizer, one or more gelling agents, and a potentiator of PDT.

27. The pharmaceutical combination of claim 26, wherein the potentiator of PDT is selected from one or more of the group consisting of sodium azide, sodium thiocyanate, sodium bromide and potassium iodide (KI), sodium iodide and potassium selenocyanate (KSeCN).

28. The pharmaceutical combination of claim 27, wherein the potentiator of PDT is potassium iodide.

29. The pharmaceutical composition of claim 28, wherein the potassium iodide is in an amount ranging from about 0.5% wt to about 5% wt, from about 1.0% wt to about 3% wt, or about 1.3% wt to about 2% wt.

30. The pharmaceutical composition of claim 29, wherein the potassium iodide is in an amount from about 1.66% wt.

31. The pharmaceutical composition of any one of claims 26-30, wherein the photosensitizer is selected from one or more of the group consisting of porphyrins, chlorins (HPPH; NPe6; Temoporfrin (Foscan), mTHPC)), and porphysomes as in: pyropheophorbide nanovesicles including, bacteriochlorophyll porphysomes, zinc pyropheophorbide porphysomes and pyropheophyorbide porphysomes, and chlorin-like compounds (benzoporphyrin; Verteporfin, bacteriochlorins and phthalocyanines, purpurins (tin ethyl etiopurpurin); Metalloporphyrins (Texaphyrins); Pheophorbides (TOOKAD); Protoporphyrins (Levulan, Metvix, 5-ALA (PpIX)) and nonporphyrin based photosensitizers including phenothiazinium salts such as Methylene Blue, Toluidine Blue, Nile Blue, Cyanines, hypericin and Chalcogenpyrilium dyes; PPA904; benzophenothiazinium dye EtNBS; the xanthene class of fluorescent dyes that includes fluorescein and Rose Bengal; Fullerenes (C60 fullerene coupled to polar diserinol groups or quaternary pyrrolidinium groups); Squaraogenines, BODIPY (boron-dipyrromethene) dye, Phenalenones; Hypericin, Hypocrellin, Riboflavin, Curcumin, Titanium dioxide and porfimer sodium (Photofrin®).

32. The pharmaceutical composition of claim 31, wherein the photosensitizer is porfimer sodium.

33. The pharmaceutical composition of claim 32, wherein the porfimer sodium is in an amount ranging from about 0.01% wt to about 1.0% wt; or about 0.05% wt to about 0.7% wt; or about 0.1% wt to about 0.5% wt; or about 0.15% wt to about 0.3% wt.

34. The pharmaceutical composition of claim 33, wherein the porfimer sodium is an amount ranging from 0.01% wt to 1.0% wt; or 0.05% wt to 0.7% wt; or 0.1% wt to 0.5% wt.

35. The pharmaceutical composition of claim 34, wherein the porfimer sodium is an amount ranging from 0.4% wt to 0.6% wt.

36. The pharmaceutical composition of any one of claims 26-35, wherein the gelling agent is selected from one or more of the group consisting of a, carbomer, crosslinked, polyacrylic acid, lecithin such as Lecithin-PLO, hydroxyethyl cellulose, hydroxypropyl cellulose, and hydroxypropyl methylcellulose.

37. The pharmaceutical composition of claim 36, wherein at least one gelling agent is hydroxypropyl cellulose.

38. The pharmaceutical composition of any one of claims 26-37, wherein the gelling agent is in an amount ranging from about 0.5% wt to about 3.0% wt, or from about 0.7% wt to about 2.0% wt, or from about 0.8% wt to about 1.2% wt.

39. The pharmaceutical composition of any one of claims 26-38, wherein the gelling agent is in an amount ranging from about 1% wt.

40. The pharmaceutical composition of any one of claims 26-39 wherein the pharmaceutical composition further comprises one or more permeation enhancers.

41. The pharmaceutical composition of claim 39, wherein the permeation enhancer is selected from one or more of the group consisting of propylene glycol SR, polyethylene glycol 400 SR, polyethylene glycol 300 LA, diethylene glycol monoethyl ether, dimethyl sulfoxide, and Polysorbate 80 SR.

42. The pharmaceutical composition of claim 41, wherein the one or more permeation enhancer is selected from the group consisting of diethylene glycol monoethyl ether and dimethyl sulfoxide.

43. The pharmaceutical composition of any one of claims 38-41 wherein the permeation enhancer is in an amount ranging from about 55.0% wt to about 85% wt, or about 60% wt to about 80% wt, or about 65% wt to about 75% wt.

44. The pharmaceutical composition of any one of claims 26-43, wherein the pharmaceutical composition comprises a photosensitizer, potassium iodide, a gelling agent, one or more permeation enhancers, a humectant, and a preservative.

45. The pharmaceutical composition of claim 43, wherein the pharmaceutical composition further comprises a surfactant, antioxidant.

46. The pharmaceutical composition of claim 44, wherein:
i) the humectant is in an amount ranging from about 10% wt to about 20% wt, or about 15% wt;
ii) the preservative is in an amount ranging from 0.5% wt to about 5% wt, or about 0.5% wt to about 2% wt, or about 1% wt to about 3% wt.

47. The pharmaceutical composition of claim 45, wherein:
i) the surfactant is in an amount ranging from about 1% wt to about 3% wt, or about 2% wt; and/or
ii) the antioxidant is in an amount ranging from 0.5% wt to about 1.5% wt, or about 1.0% wt.

48. The composition of any one of claims 26-47, further comprising a solvent.

49. The composition of claim 48, wherein the solvent is in an amount ranging from about 0.5% wt to about 1.5% wt, or about 1% wt;

50. The pharmaceutical composition of any one of claims 26-46, wherein the pharmaceutical composition is a topical formulation.

51. The pharmaceutical composition of any one of claims 46-50, wherein the composition, after 40° C./75% RH for 4 weeks, has a total impurities less than 20% by HPLC.

52. A method for treating sun exposed skin in a subject, comprising administering to the subject in need thereof any pharmaceutical composition of any one of claims 1-51, wherein the composition is applied to the sun exposed skin; and light is applied to the sun exposed skin.

53. The method of claim 52, wherein the light ranges from about 380 nm to about 850 nm in wavelength.

54. The method of claim 53, wherein the light is about 630 nm in wavelength.

55. The method of any one of claims 52-54 wherein the sun exposed skin to be treated is selected from one or more of the group consisting of chronically sun exposed skin, wrinkling, pigment spots, liver spots, actinic keratosis, seborrheic keratosis, photo-damaged skin, acne, warts, and psoriasis.

56. The method of claim 52, wherein the subject does not suffer from pain when the light is applied to the sun exposed skin.

57. The method of claim 52, wherein the subject does not suffer from pain per the visual analog scale of pain when the light is applied to the sun exposed skin.

58. The method of claim 52, wherein the subject suffers from a level pain 2, 1 or less per the visual analog scale of pain when the light is applied to the sun exposed skin.

59. The method of any one of claims 55-58, wherein the composition is applied to the skin or wound of the subject for 15 or 30 minutes, and light is applied for 5-25 minutes.

60. The method of any one of claims 55-59, wherein the composition is applied to the subject as provided in any one of Examples 6-11.

61. The method of any one of claims 55-60, wherein the subject does not develop edema or pruritis from treatment on the sun exposed skin.

62. The method of any one of claims 55-60, wherein the subject does not develop erythematous lesions greater than a score of 1 above pre-treatment from said treatment on the sun exposed skin.

63. The method of any one of claims 55-60, wherein the subject does not develop erythematous lesions higher than 1 or 2 per the erythema scale from said treatment on the sun exposed skin.

64. The method of claim 63, wherein the composition is applied to the skin or wound of the subject for 15 or 60 minutes, and light is applied for 5-25 minutes.

65. The method of claim 64, wherein the composition is applied to the subject as provided in any one of Examples 6-11.

66. The method of any one of claims 52-65, wherein the subject is treated for actinic keratosis.

67. The method of any one of claims 52-65, wherein the subject is treated for seborrheic keratosis.
